(19) United States
(12) Patent Application Publication
(10) Pub. No.: US 2024/0360076 A1
(43) Pub. Date: Oct. 31, 2024

(54) NONIONIC SURFACTANTS

(71) Applicant: Detect, Inc., Guilford, CT (US)

(72) Inventors: David N. Kamber, Guilford, CT (US); Roger R. Nani, Madison, CT (US); William A. Hansen, Clinton, CT (US)

(21) Appl. No.: 18/648,028

(22) Filed: Apr. 26, 2024

Related U.S. Application Data
(60) Provisional application No. 63/462,641, filed on Apr. 28, 2023.

(51) Int. Cl. C07C 235/08; C07C 235/34; C12Q 1/6844

(52) U.S. Cl. CPC C07C 235/08; C07C 235/34; C12Q 1/6844

(57) ABSTRACT

Standard surfactants are known to negatively impact the detection of nucleic acids in amplification and other detection techniques. Provided herein are novel surfactants that are useful in applications such as nucleic acid amplification, and that are stable under the conditions of such applications.

### RELATED APPLICATIONS

This application claims priority under 35 U.S.C. § 119 (e) to U.S. Provisional Patent Application No. 63/462,641, filed Apr. 28, 2023, which is hereby incorporated by reference in its entirety.

### GOVERNMENT SUPPORT

This invention was made with government support under 75A50122C00010 awarded by the Biomedical Advanced Research and Development Authority (BARDA). The government has certain rights in the invention.

### BACKGROUND

Fluidic devices generally require methods to help fluid flow. This is because most microfluidic and fluidic devices are assembled from materials which are hydrophobic and do not form favorable interactions with aqueous solutions. Additionally, devices with passive flow generally require surfactants or other components to help with fluid mechanics. Provided herein are small molecule surfactants that promote fluid flow.

### SUMMARY OF THE INVENTION

In one aspect, provided herein is a compound of Formula (I):

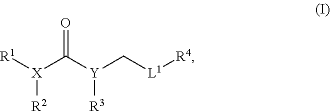

or a salt thereof, wherein:
   X and Y are each independently C or N,
   R¹ optionally substituted alkyl, or optionally substituted polyethylene glycol;
   L¹ is optionally substituted alkylene, optionally wherein one or more backbone carbon atoms in the optionally substituted alkylene are independently replaced with a moiety selected from —O—, —NR₅C(═O)—, —C(═O)NR₅—, or —NR₅—;
R² and R³ are each independently hydrogen, optionally substituted alkyl, or optionally substituted polyethylene glycol;
R⁴ is hydrogen, optionally substituted alkyl, polyethylene glycol, or optionally substituted aryl; and
R⁵ is, independently for each instance, hydrogen, optionally substituted alkyl, optionally substituted acyl, optionally substituted polyethylene glycol, or a nitrogen protecting group;
wherein the compound of formula (I) comprises at least one optionally substituted polyethylene glycol.

In another aspect of the present disclosure, provided herein is a composition comprising a compound described herein (e.g., the compound of formula (I)).

In one aspect, provided herein is a method of detecting a target nucleic acid sequence, the method comprising:
a) performing a nucleic acid amplification reaction configured to amplify the target nucleic acid sequence using a composition that comprises a compound described herein (e.g., the compound of formula (I)); and
b) detecting the presence or absence of the target nucleic acid sequence.

In another aspect of the present disclosure, provided is a kit for the detection of a target nucleic acid sequence comprising a compound described herein.

### BRIEF DESCRIPTION OF THE DRAWINGS

The accompanying drawings, which constitute a part of this specification, illustrate several embodiments of the invention and together with the description, provide non-limiting examples of the invention.

FIG. 1 shows time to positive (Tp) FluB LAMP assay data for the titration of DS2 (0%, 0.015%, 0.0575%, and 0.1%) in the presence of nasal matrix in a FluB LAMP reaction (FIG. 1A) or in LAMP buffer (FIG. 1B) (NTC is non-template control).

FIGS. 2A-B show time to positive (Tp) FluB LAMP assay data of DS2 added at 0.015% to the LAMP buffer and in the presence of nasal matrix.

FIGS. 3A-B show time to positive (Tp) FluB LAMP assay data of DS2, DS13, and DS17 added at 0.0075% to the LAMP MasterMix in the presence of nasal matrix.

FIG. 4A shows SARS-COV-2 LAMP assay data of DS2, DS13, and DS17 added at 0.0075%. FIG. 4B shows Ex2B LAMP assay data of DS2, DS13, and DS17 added at 0.0075%.

FIG. 5A shows stability data for DS2, DS13, and DS17 in amplification buffer at 8.8 pH for one month at 25° C. FIG. 5B shows stability data for DS13 from lyophilized material.

FIGS. 6A-B show photos of lyophilized LAMP components with (FIG. 6B) and without (FIG. 6A) 0.06% DS13.

FIGS. 7A-B show the time to positive (min) data for LAMP assays completed with MS2 template (0.12 cp/μL and 0.06 cp/μL) and 0.03% or 0.06% DS13.

FIGS. 8A-B show SEM images of lyophilized material with DS13 (FIGS. 8A-B).

FIG. 9 shows micelle formation data of DS1 in water. The Y-axis represents the micelle radius in nano meters and the X-axis is percent compound in solution by volume.

FIG. 10 shows micelle formation data of DS2 in water. The Y-axis represents the micelle radius in nano meters and the X-axis is percent compound in solution by volume.

FIG. 11 shows micelle formation data of DS13 in water. The Y-axis represents the micelle radius in nano meters and the X-axis is percent compound in solution by volume.

### DEFINITIONS

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7th Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3rd Edition, Cambridge University Press, Cambridge, 1987.

Compounds described herein can comprise one or more asymmetric centers, and thus can exist in various stereoisomeric forms, e.g., enantiomers and/or diastereomers. For example, in some embodiments, the compounds described herein are in the form of an individual enantiomer, diastereomer or geometric isomer, or are in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

In a formula, the bond [symbol] is a single bond, the dashed line --- is a single bond or absent, and the bond [symbol] or [symbol] is a single or double bond.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “isotopes” refers to variants of a particular chemical element such that, while all isotopes of a given element share the same number of protons in each atom of the element, those isotopes differ in the number of neutrons. The term “radioactivity” or “radioactive decay” refers to the process by which a nucleus of an unstable isotope (e.g., ¹⁸F) loses energy by emitting particles or rays (e.g., alpha particles, beta particles, and gamma rays) of ionizing radiation. Such an unstable isotope or a material including the unstable isotope is referred to as “radioactive.” The Curie (Ci) is a non-SI (non-International System of Units) unit of radioactivity and is defined as 1 Ci=3.7×10¹⁰ decays per second. The term “specific activity” refers to the unit radioactivity of a material (e.g., a compound of disclosed herein, or a salt, tautomer, stereoisomer, or isotopically labeled derivative (e.g., ¹⁸F labeled derivative) thereof). In certain embodiments, the term “specific activity” refers to the radioactivity of a material per micromole (μmol) of the material.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). In some embodiments, the one or more carbon-carbon double bonds are internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

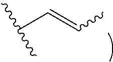

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). In some embodiments, the one or more carbon-carbon triple bonds are internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and, in some embodiments, are saturated or contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment is a carbon or nitrogen atom, as valency permits. In some embodiments, a heterocyclyl group is monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and, in some embodiments, is saturated or contains one or more carbon-carbon double or triple bonds. In some embodiments, heterocyclyl polycyclic ring systems include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, in some embodiments the point of attachment is a carbon or nitrogen atom, as valency permits. In some embodiments, heteroaryl polycyclic ring systems include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment is on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur. “Heterobiaryl” refers to an instance of two aryl rings being fused together, wherein at least one of the aryl rings is heteroaryl.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein: each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rad groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R″T groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO(C₁₋₆ alkyl), —C(═O)NH₂, —C(═O)N(C₁₋₆ alkyl)₂, OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH) OC₁₋₆ alkyl, C(═NH)N(C₁₋₆ alkyl)₂, C(═NH)NH(C₁₋₆ alkyl), C(═NH)NH₂, OC(═NH)N(C₁₋₆ alkyl)₂, OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂₀C₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, C(═O)S(C₁₋₆ alkyl), C(═S) SC₁₋₆ alkyl, —SC(═S) SC₁₋₆ alkyl, —P(═O)(OC₁₋₆ alkyl)₂, —P(═O)(C₁₋₆ alkyl)₂, —OP(═O)(C₁₋₆ alkyl)₂, OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents are joined to form ═O or ═S; and
each X⁻ is a counterion.

In some embodiments, In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb}) 2. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —C₁), bromine (bromo, —Br), or iodine (iodo, —I).

The term “hydroxyl” or “hydroxy” refers to the group —OH. The term “substituted hydroxyl” or “substituted hydroxy,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X, R^{aa}, R^{bb}, and R^{cc} are as defined herein.

The term “alkoxy” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S)N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb}) 3 and —N(R^{bb})₃⁺X⁻, wherein R^{bb} and X are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, C(═O)SR^{X1}, —C(═O)N(R^{X1})₂, —C(═S) R^{X1}, —C(═S)N(R^{X1})₂, and —C(═S)S(R^{X1}), —C(═NR^{X1})R^{X1}, C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “boronyl” refers to boranes, boronic acids, boronic esters, borinic acids, and borinic esters, e.g., boronyl groups of the formula —B(R^{aa})₂, —B(OR^{cc})₂, and —BR^{aa}(OR^{cc}), wherein R^{aa} and R^{cc} are as defined herein.

The term “phosphino” refers to the group —P(R^{cc})₂, wherein R^{cc} is as defined herein.

The term “phosphono” refers to the group —(P═O)(OR^{cc})₂, wherein R^{aa} and R^{cc} are as defined herein.

The term “phosphoramido” refers to the group —O(P═O)(N(R^{bb})₂)₂, wherein each R^{bb} is as defined herein.

The term “stannyl” refers to the group —Sn(R^{cc})₃, wherein R^{cc} is as defined herein.

The term “germyl” refers to the group —Ge(R^{cc})₃, wherein R^{cc} is as defined herein.

The term “arsenyl” refers to the group —As(R^{cc})₂, wherein R^{cc} is as defined herein.

The term “oxo” refers to the group ═O, and the term “thiooxo” refers to the group ═S.

In some embodiments, nitrogen atoms are substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{ee} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N-(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten) acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include-R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, 4,4′-dimethoxytrityl (4,4′-dimethoxytriphenylmethyl or DMT), α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3″′-[N-(imidazolylethyl) carbamoyl]trityl Ether (IETr-OR), 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. In some embodiments, a leaving group is an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O) 2N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N, O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of the present disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “stoichiometric solvate” refers to a solvate, which comprises a compound (e.g., a compound disclosed herein) and a solvent, wherein the solvent molecules are an integral part of the crystal lattice, in which they interact strongly with the compound and each other. The removal of the solvent molecules will cause instability of the crystal network, which subsequently collapses into an amorphous phase or recrystallizes as a new crystalline form with reduced solvent content.

The term “non-stoichiometric solvate” refers to a solvate, which comprises a compound (e.g., a compound disclosed herein) and a solvent, wherein the solvent content may vary without major changes in the crystal structure. The amount of solvent in the crystal lattice only depends on the partial pressure of solvent in the surrounding atmosphere. In the fully solvated state, non-stoichiometric solvates may, but not necessarily have to, show an integer molar ratio of solvent to the compound. During drying of a non-stoichiometric solvate, a portion of the solvent may be removed without significantly disturbing the crystal network, and the resulting solvate can subsequently be resolvated to give the initial crystalline form. Unlike stoichiometric solvates, the desolvation and resolvation of non-stoichiometric solvates is not accompanied by a phase transition, and all solvation states represent the same crystal form.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgaard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C2-C8 alkenyl, C2-C8 alkynyl, aryl, C7-C12 substituted aryl, and C7-C12 arylalkyl esters of the compounds described herein may be preferred.

The term “critical micelle concentration” (CMC) as used herein defines the concentration of a compound in solution which induces the formation of micelles. In some embodiments, the CMC is a function of temperature. In some embodiments, the CMC increases with temperature. In some embodiments, the CMC varies in different solvent conditions.

As used herein, the term “stable” is defined as a compounds ability to maintain its chemical purity under certain conditions. In some embodiments, the compound is stable when exhibiting a purity of ≥80% following one month of storage at between 15-30° C. in a solution with a pH of 8.8. In some embodiments, the compound is stable when exhibiting a purity of ≥90% following one month of storage at between 15-30° C. in a solution with a pH of 8.8. In some embodiments, the compound is stable when exhibiting a purity of ≥95% following one month of storage at between 15-30° C. in a solution with a pH of 8.8. In some embodiments, the compound is stable when exhibiting a purity of ≥80% following one month of storage at 40° C. In some embodiments, the compound is stable when exhibiting a purity of ≥90% following one month of storage at 40° C. In some embodiments, the compound is stable when exhibiting a purity of ≥95% following one month of storage at 40° C. In some embodiments, the compound is stable when exhibiting a purity of ≥80% following two months of storage at 40° C. In some embodiments, the compound is stable when exhibiting a purity of ≥90% following two months of storage at 40° C. In some embodiments, the compound is stable when exhibiting a purity of ≥95% following two months of storage at 40° C. The term “shelf-stable” refers to a compound maintaining purity of >70% at 15-30° C. in aqueous solution for an extended amount of time. In some embodiments, the extended amount of time is longer than one month.

The term “monodispersed” indicates a compound with uniform size or molecular weight within a mixture or dispersed in solution.

The term “crude sample matrix” as used herein represents a sample which is collected from a source (i.e., a mammal, or tissue culture) which has not undergone purification. In some embodiments, a crude sample matrix is a nasal swab, mucus, saliva, sputum, urine, blood, or cell scraping sample. In some embodiments, a crude sample matrix is a nasal swab. In some embodiments, a crude sample matrix is blood. In some embodiments, a crude sample matrix is urine. In some embodiments, a crude sample matrix is blood. In some embodiments, a crude sample matrix is a biological sample.

As used herein, the term “wettability” refers to the ability of a substance or solute to interact with water. Increased wettability (or increased wetting) correlates to an increase in hydrophilicity and therefor more favorable interactions between the solute or substance and water. Decreased wettability correlates to an increase in hydrophobicity and therefor less favorable interactions between the solute or substance and water.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values

Unless otherwise required by context, singular terms shall include pluralities, and plural terms shall include the singular.

These and other exemplary substituents are described in more detail in the Detailed Description, Examples, and Claims. The present disclosure is not limited in any manner by the above exemplary listing of substituents.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonuculeotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N₆-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N₆-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

### DETAILED DESCRIPTION OF CERTAIN EMBODIMENTS

Surfactants are amphiphilic, with polar and hydrophobic functional groups, the polar head group can form intermolecular interactions with aqueous solutions and suppress hydrophobic cavitation on material surfaces. As a result, surfactants help fluid flow by decreasing the surface tension and promoting wetting of aqueous solutions on hydrophobic surfaces.

Design of surfactants requires attention to the length of the hydrophobic chain and the identity of the polar head group, which may also influence the formation of micelles in solutions. The formation of micelles may influence the wettability on hydrophobic surfaces, but formation of micelles is generally detrimental to membrane integrity of cells. For example, surfactants with long, linear aliphatic carbon chains generally have a lower critical micelle concentration (CMC) relative to surfactants with shorter aliphatic carbon chains.

In some embodiments, the disclosed surfactants are designed with one or more PEG chains attached through one or more amide groups to one or more aliphatic carbon chains with variable lengths.

There are many other surfactants commonly used to promote fluid flow in devices, including nonionic surfactants (e.g. Tween20, Triton-X-100), ionic surfactants (e.g. SDS), non ionic triblock copolymers (e.g. Pluronic F68), cationic surfactants (e.g. CTAB). However, these alternative surfactants have one or more drawbacks, notably:
1. Lack of spectral signature to facilitate simple, fast analytical measurements of surfactant concentration and integrity.
2. Polydispersity (e.g. in the case of triblock co-polymers) that results in high variability in material composition between independently manufactured batches.
3. Lack of stability when stored in aqueous solution at ambient temperature.
4. Lack of compatibility with biological reagents and/or reactions, including for example, nucleic acid amplification reactions such as loop mediated isothermal amplification (LAMP).
5. Promote degradation of nucleic acid (especially RNA) present in a crude sample (e.g. human nasal swab) when such a crude sample is exposed to the surfactant and heated to above-ambient temperatures (possible by altering the integrity of cells and/or microbes)

In some embodiments, the disclosed novel small molecule surfactants address one or more of these drawbacks by:
1. Promoting fluid flow in fluidic devices, including over hydrophobic materials
2. Containing an amide bond that characteristically absorbs UV light between 210-230 nm facilitating both surfactant purification as well as subsequent measurements to confirm surfactant integrity and concentration
3. Being monodispersed
4. Having stability in aqueous solution
5. being compatible with loop mediated isothermal amplification (LAMP) and the biological reagents contained therein (e.g. polymerase, reverse transcriptase).
6. Not promoting degradation of nucleic acids upon heating a crude sample containing the nucleic acid target of interest and surfactant to above-ambient temperatures

In certain embodiments, the disclosed surfactants may be suitable for use in any application where a surfactant is commonly applied (e.g., reduce surface tension, emulsify oils/dirt, enhance wetting and spreading, solubilize/disperse/stabilize molecules of interest, serve as a foaming agent).

In some embodiments, the disclosed surfactant may be used in a diagnostic test system. In certain embodiments, the diagnostic testing system is a multi-step system. In some embodiments, the diagnostic system comprises one or more of the following: collects a test sample, treats the sample in a manner that prepares it for subsequent analysis, directs sample flow through the device, or performs one or more operations that result in qualitative and/or quantitative detection of one or more analytes of interest. In some embodiments, the compound promotes fluid flow over device materials. In some embodiments, the compound is compatible with the biological/chemical reactions occurring in the device. In some embodiments, the compound is shelf stable.

Recently, Detect has realized a fluidic cartridge for use in a rapid, low cost molecular diagnostic test that involves detecting one or more analytes from a collected sample. A step in the workflow of such tests is an optional sample preparation step that employs above-ambient heating (e.g. 50-95° C.) that alters the properties of a collected sample matrix in such a way that improves subsequent detection of nucleic acid present in that matrix (i.e., mucous nasal matrix). It is hypothesized that heat may be beneficial by promoting a decrease in sample viscosity; facilitating cellular/microbial lysis; or cleaving, digesting, or inactivating a protein, glycan, or proteoglycan (e.g., an interfering enzyme) present in the matrix.

Although heat is beneficial for sample preparation, it has been demonstrated (described in US-2022-0305496-A1) that applying heat to a crude biological sample that contains a target nucleic acid (e.g. RNA or DNA) molecule of interest and a conventional surfactant severely impairs subsequent detection of the target nucleic acid. It is hypothesized that conventional surfactants promote degradation of RNA molecules through an unknown mechanism in the presence of crude samples and heat-possibly by affecting microbial/cellular integrity. Other diagnostic test systems involve a complex nucleic acid purification step that avoids combining surfactant, heat and crude sample matrix. However, Detect's cartridge design avoids such complex biochemical operations to improve usability, reduce test cost, decrease test time to result, and/or avoid the use of toxic/harsh chemicals.

The disclosed compounds are thus highly inventive and unique in their ability to provide the conventional benefits of a surfactant (i.e. enhanced wettability and spreading, reduce surface tension in a diagnostic device containing fluidic channels), while enabling a simple sample preparation workflow where heat is used to resolve a sample matrix that contains a target nucleic acid, retaining compatibility with subsequent nucleic acid amplification steps, and having a long shelf life when stored in aqueous solution at ambient temperature.

In some embodiments, the disclosed compounds have utility in cleaning products (e.g., detergents, soaps, disinfectants), personal care products (e.g., shampoos, body washes, lotions), agriculture (e.g., wetting agents, adjuvants to help pesticides or herbicides better adhere to plant surfaces and create more effective and eco-friendly agricultural products protected from environmental factors), oil recovery (e.g., recovery of oil from reservoirs or spills by reducing the surface tension between oil and water) or the pharmaceutical industry (e.g., drug delivery agents, solubility or bioavailability enhancement).

The disclosed compounds are novel compositions of matter. In another aspect, provided herein is a method of using the compound in a diagnostic test system. Methods for using such compounds in fluid flow and compatibility with LAMP reaction has not been disclosed before.

In some embodiments, the disclosed compound improves fluid mechanics in microfluidic devices (e.g., devices produced from Zeonor and PMMA materials), improves fluid mechanics in devices with passive fluidic flow, and is compatible with nucleic acid amplification reactions such as loop mediated isothermal amplification (LAMP). In some embodiments, the disclosed compound improves fluid mechanics in microfluidic devices (e.g., devices produced from Zeonor and PMMA materials). In some embodiments, the disclosed compound improves fluid mechanics in devices with passive fluidic flow. In some embodiments, the disclosed compound is compatible with nucleic acid amplification reactions such as loop mediated isothermal amplification (LAMP).

List of unsuccessful surfactants screened for fluid flow in Zeonor cartridges and compatibility with LAMP: Tween 20, EcoSurf SA-9, EcoSurf EH-3, Empigen 88, Triton CG 110, LDAO, Fos-choline-12, CHAPS, m-PEG₃₆-alcohol, Urea, Betaine, Decyl-β-D-Glucopyranoside, Dodecyl-β-D-Maltopyranoside.

In some embodiments, the disclosed compounds comprise one or more PEG chains attached through one or more amide groups to one or more aliphatic carbon chains with variable lengths.

In some embodiments, the compound is of the formula:

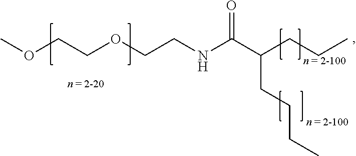

or a salt thereof.

In some embodiments, the compound is of the formula:

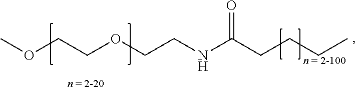

or a salt thereof.

In some embodiments, the compound is of the formula:

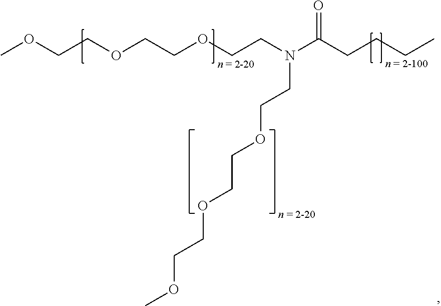

or a salt thereof.

In some embodiments, the compound is of the formula:

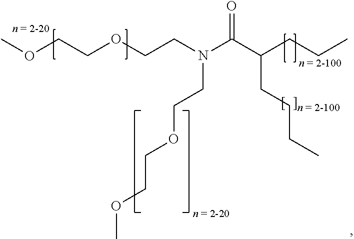

or a salt thereof.

In some embodiments, the compound is of the formula:

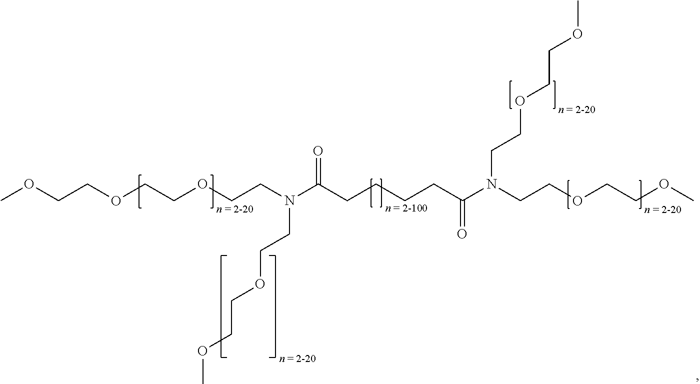

or a salt thereof.

In some embodiments, the compound is of the formula:

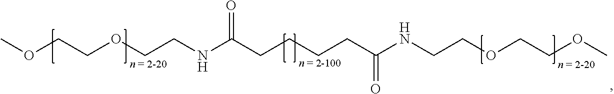

or a salt thereof.

In some embodiments, the compound is of the formula:

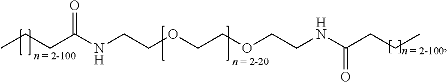

or a salt thereof.

In one aspect of the present disclosure, provided herein a compound of Formula (I):

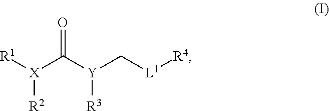

or pharmaceutically acceptable salt, solvate, hydrate, polymorph, co-crystal, tautomer, stereoisomer, isotopically labeled compound, or prodrug thereof, wherein:
   X and Y are each independently C or N,
   R¹ optionally substituted alkyl, or optionally substituted polyethylene glycol;
   L¹ is optionally substituted alkylene, optionally wherein one or more backbone carbon atoms in the optionally substituted alkylene are independently replaced with a moiety selected from —O—, —NR₅C(═O)—, —C(═O)NR₅—, or —NR₅—;
   R² and R³ are each independently hydrogen, optionally substituted alkyl, or optionally substituted polyethylene glycol;
   R⁴ is independently hydrogen, optionally substituted alkyl, polyethylene glycol, or optionally substituted aryl; and
   R⁵ is hydrogen, optionally substituted alkyl, optionally substituted acyl, optionally substituted polyethylene glycol, or a nitrogen protecting group;
   wherein the compound of formula (I) comprises at least one optionally substituted polyethylene glycol.

In some embodiments, X is C. In certain embodiments, X is N. In some embodiments, Y is C. In some embodiments, Y is N. In some embodiments, X is N and Y is C. In some embodiments, X is C and Y is N. In some embodiments, X is C.

In some embodiments, the compound is of formula (I-a):

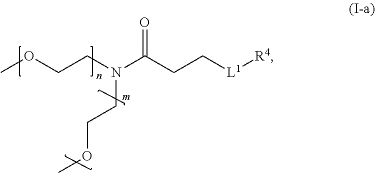

or pharmaceutically acceptable salt, solvate, hydrate, polymorph, co-crystal, tautomer, stereoisomer, isotopically labeled compound, or prodrug thereof, wherein: n and m are each independently 1-20.

In certain embodiments, n is 1-10. In some embodiments, n is 1, 2, 3, 4, 5, 6, 7 or 8. In some embodiments, n is 2. In some embodiments, n is 3. In some embodiments, n is 4. In some embodiments, n is 5. In some embodiments, n is 6. In certain embodiments n is 7. In certain embodiments, n is 8.

In certain embodiments, m is 1-10. In some embodiments, m is 1, 2, 3, 4, 5, 6, 7 or 8. In some embodiments, m is 2. In some embodiments, m is 3. In some embodiments, m is 4. In some embodiments, m is 5. In some embodiments, m is 6. In certain embodiments m is 7. In certain embodiments, m is 8.

In some embodiments, the compound is of formula (I-a), or pharmaceutically acceptable salt, solvate, hydrate, polymorph, co-crystal, tautomer, stereoisomer, isotopically labeled compound, or prodrug thereof, wherein n is 1-20; m is 1-20; L¹ is optionally substituted alkylene, optionally wherein one or more backbone carbon atoms in the optionally substituted alkylene are independently replaced with a moiety selected from —O—, —NR₅C(═O)—, —C(═O)NR₅—, or —NR₅—; and R⁴ is hydrogen, optionally substituted alkyl, polyethylene glycol, or optionally substituted aryl.

In some embodiments, the compound is of formula (I-a), or pharmaceutically acceptable salt, solvate, hydrate, polymorph, co-crystal, tautomer, stereoisomer, isotopically labeled compound, or prodrug thereof, wherein n is 1-10; m is 1-10; L¹ is optionally substituted alkylene, wherein one or more backbone carbon atoms in the optionally substituted alkylene are independently replaced with a moiety selected from —O—, —NR₅C(═O)—, —C(═O)NR₅—, or —NR₅—; and R⁴ is hydrogen, optionally substituted alkyl, polyethylene glycol, or optionally substituted aryl.

In some embodiments, the compound is of formula (I-a), or pharmaceutically acceptable salt, solvate, hydrate, polymorph, co-crystal, tautomer, stereoisomer, isotopically labeled compound, or prodrug thereof, wherein n is 3 or 4; m is 3 or 4; L¹ is C₁₋₁₂ alkylene, wherein one or more backbone carbon atom in the C₁₋₁₂ alkylene is replaced with a moiety selected from —O—, —NR₅C(═O)—, —C(═O)NR₅—, or —NR₅—; and R⁴ is hydrogen, optionally substituted alkyl, polyethylene glycol, or optionally substituted aryl.

In certain embodiments, the compound is of formula (I-a-I):

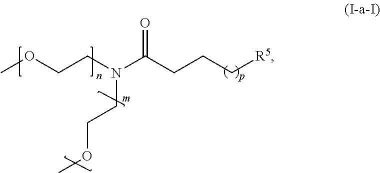

or pharmaceutically acceptable salt, solvate, hydrate, polymorph, co-crystal, tautomer, stereoisomer, isotopically labeled compound, or prodrug thereof, wherein: p is 1-20; and R⁵ is hydrogen or phenyl.

In some embodiments, p is 11-20. In some embodiments, p is 11, 12, 13, 14, 15, 16, 17, 18, 19, or 20. In some embodiments, p is 1-10. In some embodiments, p is 3-15. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10.

In certain embodiments, R⁵ is hydrogen. In some embodiments, R⁵ is phenyl.

In certain embodiments, the compound is of formula (I-a-I) wherein p is 1-12, and R⁵ is hydrogen. In certain embodiments, the compound is of formula (I-a-I) wherein p is 1-12, and R⁵ is phenyl.

In certain embodiments, the compound is of formula (I-a-I) wherein p is 1, and R⁵ is hydrogen. In certain embodiments, the compound is of formula (I-a-I) wherein p is 1, and R⁵ is phenyl.

In certain embodiments, the compound is of formula (I-a-I) wherein p is 3, and R⁵ is hydrogen. In certain embodiments, the compound is of formula (I-a-I) wherein p is 3, and R⁵ is phenyl.

In certain embodiments, the compound is of formula (I-a-I) wherein p is 5, and R⁵ is hydrogen. In certain embodiments, the compound is of formula (I-a-I) wherein p is 5, and R⁵ is phenyl.

In certain embodiments, the compound is of formula (I-a-I) wherein p is 6, and R⁵ is hydrogen. In certain embodiments, the compound is of formula (I-a-I) wherein p is 6, and R⁵ is phenyl.

In certain embodiments, the compound is of formula (I-a-I) wherein p is 8, and R$ is hydrogen. In certain embodiments, the compound is of formula (I-a-I) wherein p is 8, and R⁵ is phenyl.

In certain embodiments, the compound is of formula (I-a-I) wherein p is 12, and R⁵ is hydrogen. In certain embodiments, the compound is of formula (I-a-I) wherein p is 12, and R⁵ is phenyl.

In certain embodiments, the compound is of formula (I-a-I) wherein p is 1-12, and R⁵ is hydrogen or phenyl, and m and n are each independently 3 or 4. In certain embodiments, the compound is of formula (I-a-I) wherein p is 1-12, and R⁵ is hydrogen, and m and n are each independently 3 or 4. In certain embodiments, the compound is of formula (I-a-I) wherein p is 1-12, and R⁵ is phenyl, and m and n are each independently 3 or 4.

In certain embodiments, the compound is of formula (I-a-I) wherein p is 12, and R⁵ is hydrogen or phenyl, and m and n are each independently 3 or 4.

In certain embodiments, n and m are each independently 3 or 4. In certain embodiments, n is 3 and m is 4. In certain embodiments, n is 3 and m is 3. In certain embodiments, n is 4 and m is 3. In some embodiments n is 4 and m is 4.

In certain embodiments, the compound is of formulae (I-a-II), (I-a-III), (I-a-IV), or (I-a-V):

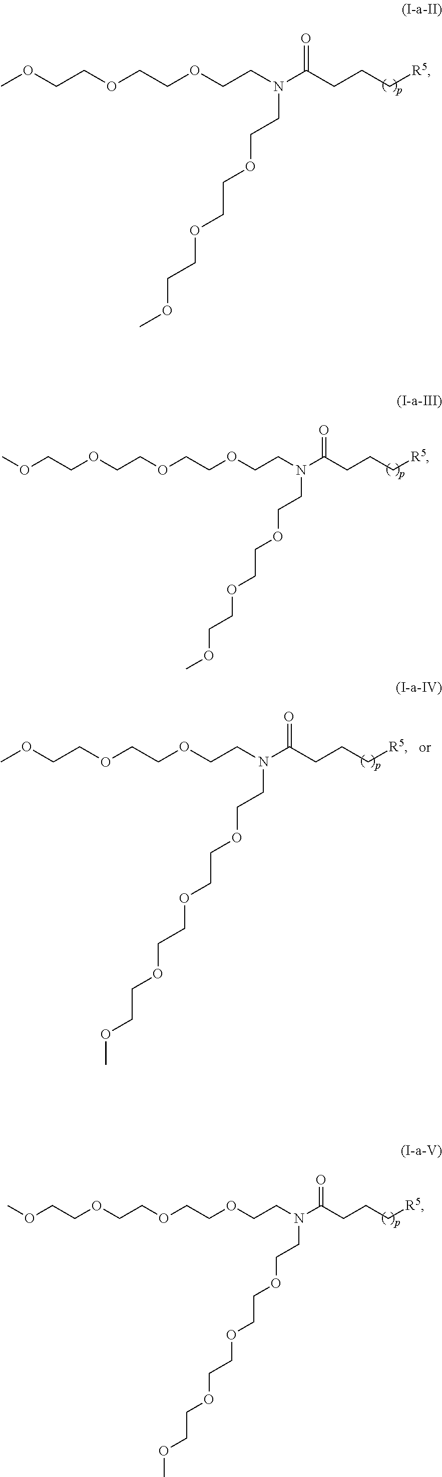

or pharmaceutically acceptable salt, solvate, hydrate, polymorph, co-crystal, tautomer, stereoisomer, isotopically labeled compound, or prodrug thereof.

In certain embodiments, the compound is of formula (I-a-II), wherein p is 1-15 and R⁵ is hydrogen. In certain embodiments, the compound is of formula (I-a-II), wherein p is 1-15 and R⁵ is phenyl. In some embodiments, the compound is of formula (I-a-III), wherein p is 1-15 and R⁵ is phenyl. In some embodiments, the compound is of formula (I-a-III), wherein p is 1-15 and R⁵ is hydrogen. In some embodiments, the compound is of formula (I-a-IV), wherein p is 1-15 and R⁵ is hydrogen. In some embodiments, the compound is of formula (I-a-IV), wherein p is 1-15 and R⁵ is phenyl. In some embodiments, the compound is of formula (I-a-V), wherein p is 1-15 and R⁵ is hydrogen. In some embodiments, the compound is of formula (I-a-V), wherein p is 1-15 and R⁵ is phenyl.

In certain embodiments, the compound is

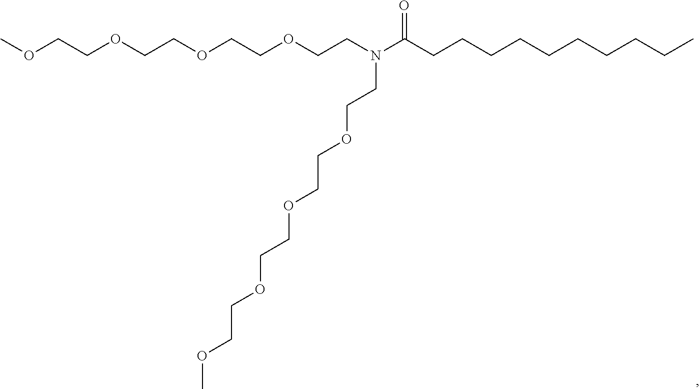 [SMILES: CCCCCCCCCCC(=O)N(CCOCCOCCOCCOC)CCOCCOCCOCCOC]

or pharmaceutically acceptable salt, solvate, hydrate, polymorph, co-crystal, tautomer, stereoisomer, isotopically labeled compound, or prodrug thereof.

In some embodiments, the compound is of formula (I-a-V):

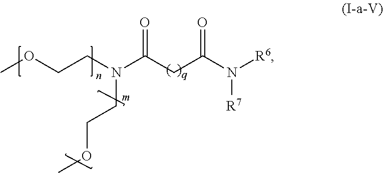

or pharmaceutically acceptable salt, solvate, hydrate, polymorph, co-crystal, tautomer, stereoisomer, isotopically labeled compound, or prodrug thereof, wherein: R⁶ and R⁷ are each optionally substituted polyethylene glycol and q is 1-20.

In some embodiments, the compound is of formula (I-a-V) wherein R⁶ and R⁷ are each polyethylene glycol and q is 1-20. In some embodiments, the compound is of formula (I-a-V) wherein R⁶ and R⁷ are each polyethylene glycol and q is 1-10. In some embodiments, the compound is of formula (I-a-V) wherein R⁶ and R⁷ are each polyethylene glycol and q is 8.

In certain embodiments, the compound is of formula (I-b):

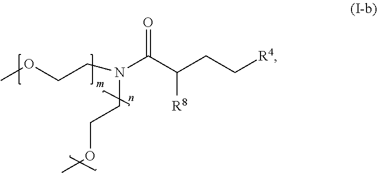

or pharmaceutically acceptable salt, solvate, hydrate, polymorph, co-crystal, tautomer, stereoisomer, isotopically labeled compound, or prodrug thereof, wherein: R⁸ is optionally substituted C₁₋₂₀ alkyl.

In certain embodiments, the compound is of formula (I-b) wherein R⁸ is optionally substituted C₁₋₂₀ alkyl and R⁴ is optionally substituted alkyl, polyethylene glycol, or optionally substituted aryl. In certain embodiments, the compound is of formula (I-b) wherein R⁸ is C₁₋₂₀ alkyl and R⁴ is C₁₋₂₀ alkyl. In certain embodiments, the compound is of formula (I-b) wherein R⁸ is C₁₋₆ alkyl and R⁴ is C₁₋₆ alkyl. In some embodiments, R⁴ and R⁸ are each unsubstituted C₁₋₆ alkyl.

In certain embodiments, the compound is of formula (I-c):

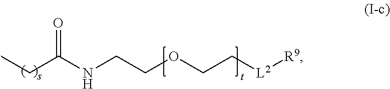

or pharmaceutically acceptable salt, solvate, hydrate, polymorph, co-crystal, tautomer, stereoisomer, isotopically labeled compound, or prodrug thereof, wherein:
   s and t are each independently 1-20;
   L² is a bond or optionally substituted alkylene, optionally wherein one or more backbone carbon atoms in the optionally substituted alkylene are independently replaced with a moiety selected from —O—, —NR₅C(═O)—, —C(═O)NR₅—, or —NR₅—; and
   R⁹ is hydrogen, methyl, or optionally substituted aryl.

In certain embodiments, the compound is of formula (I-c) wherein s and t are each independently 1-10; L² is a bond or optionally substituted alkylene, optionally wherein one or more backbone carbon atoms in the optionally substituted alkylene are independently replaced with a moiety selected from —O—, —NR₅C(═O)—, —C(═O)NR₅—, or —NR₅—; and R⁹ is hydrogen, methyl, or optionally substituted aryl.

In certain embodiments, the compound is of formula (I-c) wherein s and t are each independently 1-10; L² is a bond or optionally substituted alkylene, optionally wherein one or more backbone carbon atoms in the optionally substituted alkylene are independently replaced with a moiety selected from —O—, —NR₅C(═O)—, —C(═O)NR₅—, or —NR₅—; and R⁹ is hydrogen, or methyl.

In some embodiments, the compound is of formula (I-c-I):

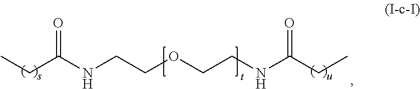

or pharmaceutically acceptable salt, solvate, hydrate, polymorph, co-crystal, tautomer, stereoisomer, isotopically labeled compound, or prodrug thereof, wherein: u is 1-20.

In some embodiments, the compound is of formula (I-c-I), wherein u is 1-20, sis 1-20, and t is 1-10. In some embodiments, s and u are each independently 1-10. In certain embodiments, s and u are each independently 4 or 9. In some embodiments, the compound is of formula (I-c-I), wherein u is 1-10, sis 1-15, and t is 1-10. In some embodiments, the compound is of formula (I-c-I), wherein u is 4, s is 1-15, and t is 1-10. In some embodiments, the compound is of formula (I-c-I), wherein u is 9, s is 1-15, and t is 1-10. In some embodiments, the compound is of formula (I-c-I), wherein u is 1-10, s is 1-15, and t is 4. In some embodiments, the compound is of formula (I-c-I), wherein u is 1-10, sis 1-15, and t is 9. In some embodiments, the compound is of formula (I-c-I), wherein u is 4, s is 1-15, and t is 4. In some embodiments, the compound is of formula (I-c-I), wherein u is 9, s is 1-15, and t is 4. In some embodiments, the compound is of formula (I-c-I), wherein u is 4, s is 1-15, and t is 9. In some embodiments, the compound is of formula (I-c-I), wherein u is 9, sis 1-15, and t is 9.

In some embodiments, t is 1-10. In certain embodiments, t is 2, 7, or 10. In certain embodiments, t is 2. In certain embodiments, t is 7. In some embodiments, t is 10.

In some embodiments, compound is of formula (I-c-II):

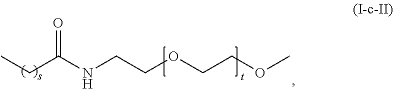

or pharmaceutically acceptable salt, solvate, hydrate, polymorph, co-crystal, tautomer, stereoisomer, isotopically labeled compound, or prodrug thereof.

In certain embodiments, s is 1-10. In some embodiments, s is 4 or 9. In certain embodiments, t is 1-10. In some embodiments, t is 5 or 7.

In some embodiments, compound is of formula (I-c-II), wherein s and t are each independently 1-10. In some embodiments, compound is of formula (I-c-II), wherein s is 9 and t is 1-10. In some embodiments, compound is of formula (I-c-II), wherein s is 9 and t is 5-10. In some embodiments, compound is of formula (I-c-II), wherein s is 4 and t is 1-10. In some embodiments, compound is of formula (I-c-II), wherein s is 4 and t is 5-10.

In certain embodiments, the compound is of formula (I-c-III):

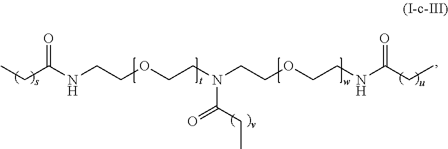

or pharmaceutically acceptable salt, solvate, hydrate, polymorph, co-crystal, tautomer, stereoisomer, isotopically labeled compound, or prodrug thereof, wherein: v and w are each independently 1-20.

In some embodiments, s, u, and v are each independently 1-10. In some embodiments, s, u, and v are 9.

In some embodiments, t and w are each independently 1-10. In some embodiments, t and w are each independently 1-5. In some embodiments, t and w are 4.

In certain embodiments, the compound is of formula (I-c-III), wherein, s, t, u, v and w are each independently 1-10. In certain embodiments, the compound is of formula (I-c-III), wherein, s, v, and u are 9 and t, and w are each independently 1-5. In certain embodiments, the compound is of formula (I-c-III), wherein, s, v, and u are 4-10, t and w are each 4.

In some embodiments, the compound is:

| Compound | Structure |
|---|---|
| DS1 | 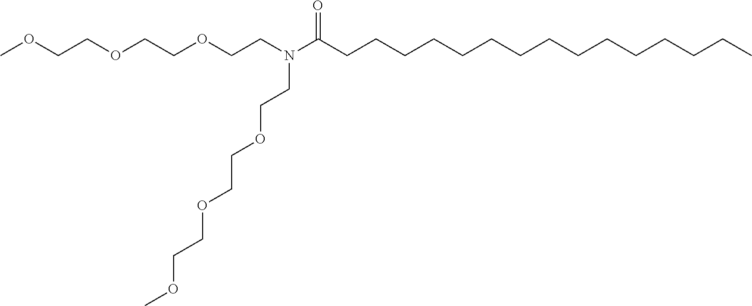 [SMILES: CCCCCCCCCCCCCCCC(=O)N(CCOCCOCCOC)CCOCCOCCOC] |
| DS2 | 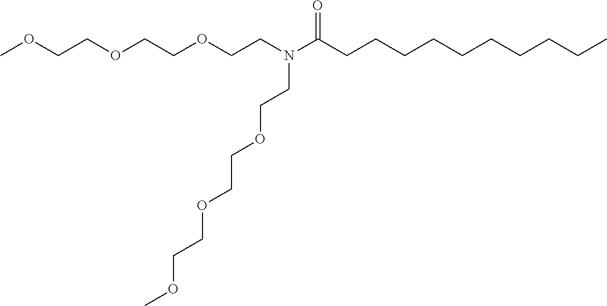 [SMILES: CCCCCCCCCCC(=O)N(CCOCCOCCOC)CCOCCOCCOC] |
| DS3 | 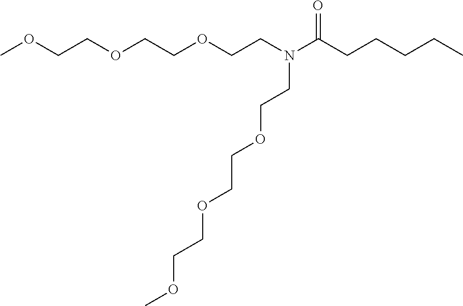 [SMILES: CCCCCC(=O)N(CCOCCOCCOC)CCOCCOCCOC] |
| DS4 | 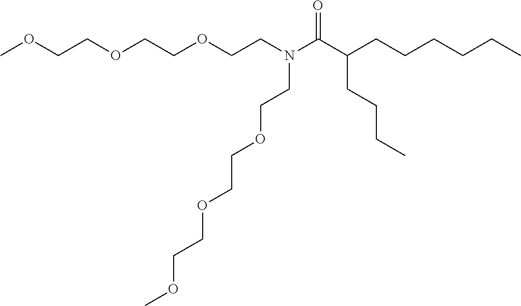 [SMILES: CCCCCCC(CCCC)C(=O)N(CCOCCOCCOC)CCOCCOCCOC] |
| DS5 | 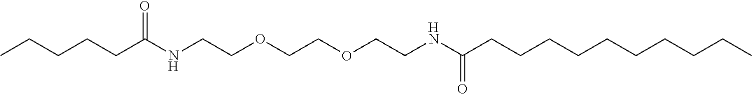 |
| DS6 | 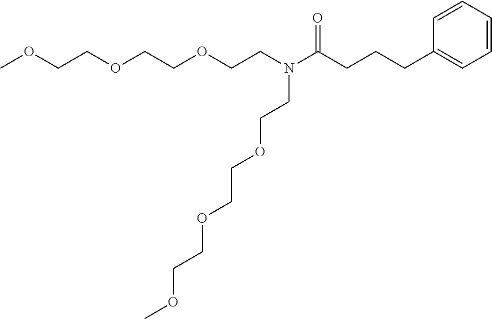 [SMILES: COCCOCCOCCN(CCOCCOCCOC)C(=O)CCCc1ccccc1] |
| DS7 | 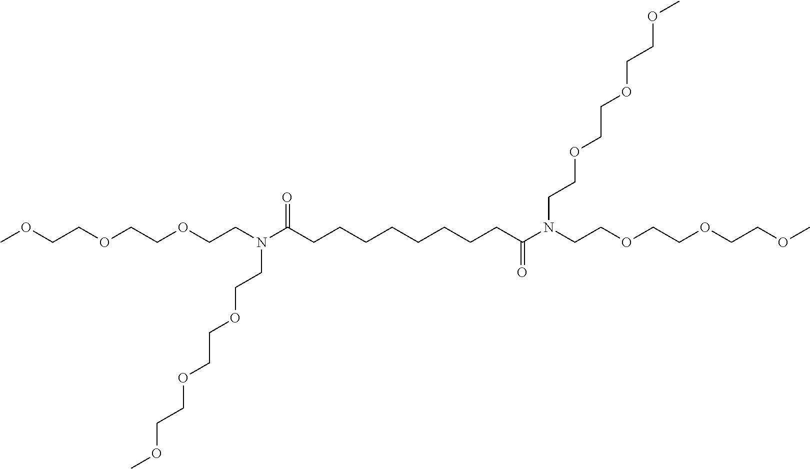 |
| DS8 | 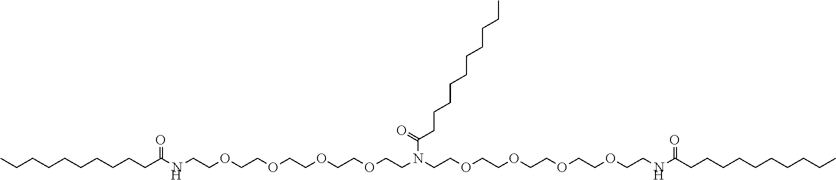 |
| DS9 | 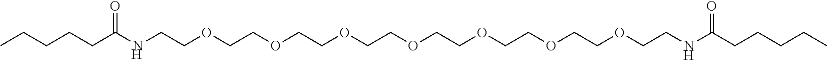 |
| DS10 | 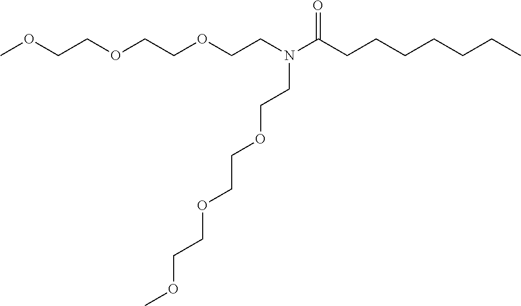 [SMILES: CCCCCCCC(=O)N(CCOCCOCCOC)CCOCCOCCOC] |
| DS11 | 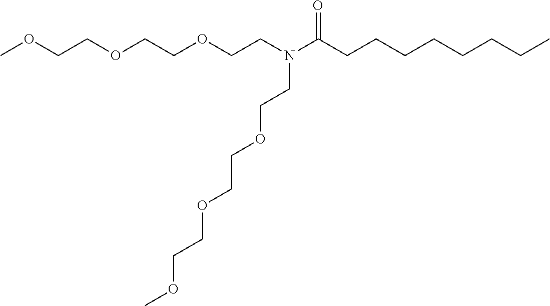 [SMILES: CCCCCCCCC(=O)N(CCOCCOCCOC)CCOCCOCCOC] |
| DS12 | 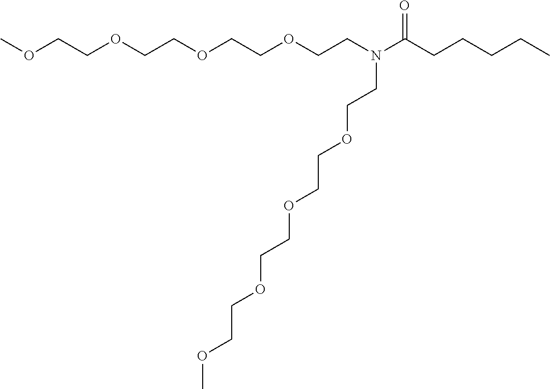 [SMILES: CCCCCC(=O)N(CCOCCOCCOCCOC)CCOCCOCCOCCOC] |
| DS13 | 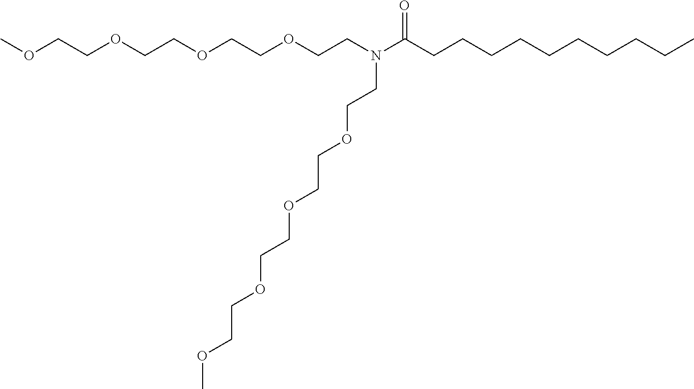 [SMILES: CCCCCCCCCCC(=O)N(CCOCCOCCOCCOC)CCOCCOCCOCCOC] |
| DS14 | 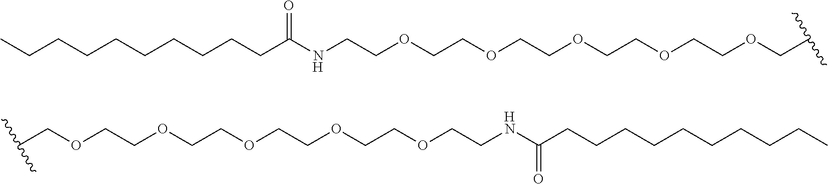 |
| DS15 | 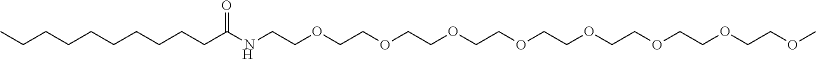 |
| DS16 | 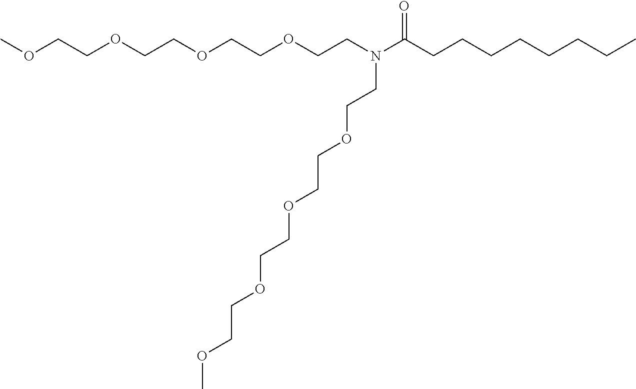 [SMILES: CCCCCCCCC(=O)N(CCOCCOCCOCCOC)CCOCCOCCOCCOC] |
| DS17 | 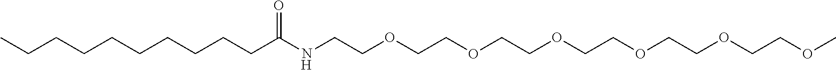 |
| DS18 | 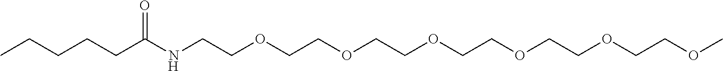 |

or pharmaceutically acceptable salt, solvate, hydrate, polymorph, co-crystal, tautomer, stereoisomer, isotopically labeled compound, or prodrug thereof.

In certain embodiments, the compound does not degrade nucleic acids. In certain embodiments, the compound is compatible with nucleic acid amplification techniques. In certain embodiments, the compound does not interfere with nucleic acid detection techniques. In certain embodiments, the compound does not interfere with nucleic acid amplification techniques. In certain embodiments, the nucleic acid amplification technique is loop-mediated isothermal amplification (LAMP). In certain embodiments, the compound is compatible with LAMP reagents. In some embodiments, the LAMP reagents are selected from polymerase enzymes, reverse transcriptase enzymes, nucleic acids (e.g., deoxyribonucleotide triphosphates (dNTPs)) excipients, primers, and buffers.

In some embodiments, the compound is compatible with LAMP conditions. In certain embodiments, the LAMP conditions are at temperatures of between 50-95° C.

In certain embodiments, the compound has a critical micelle concentration (CMC) of greater than or equal to 0.005%. In some embodiments, the compound has a critical micelle concentration (CMC) of greater than or equal to 0.1%. In certain embodiments, the compound has a critical micelle concentration (CMC) of 0.005-0.5%. In certain embodiments, the compound has a critical micelle concentration (CMC) of 0.1-0.5%. In certain embodiments, the compound has a critical micelle concentration (CMC) of greater than or equal to 0.3%. In certain embodiments, the compound has a critical micelle concentration (CMC) of 0.3%.

In certain embodiments, compound is stable at room temperature. In some embodiments, the compound is stable in aqueous solution. In some embodiments, the compound is stable in aqueous solution at between, 14-30° C. In some embodiments, the compound is stable in aqueous solution at between, 14-30° C. for more than one month. In some embodiments, the compound is stable in aqueous solution at 40° C. In some embodiments, the compound is stable in aqueous solution at 40° C. for more than one month. In some embodiments, the compound is stable in aqueous solution at a basic pH. In some embodiments, the compound is stable in aqueous solution at pH 8.8. In some embodiments, the compound is stable in aqueous solution at pH 8.8 at between, 14-30° C. In some embodiments, the compound is stable in aqueous solution at pH 8.8 at 40° C.

In another aspect, provided herein is a composition comprising the compound disclosed herein.

In certain embodiments, the composition is characterized as being monodisperse.

In some embodiments, the composition further comprises a test sample. In certain embodiments, the test sample is animal bodily tissue or fluid. In certain embodiments, the test sample comprises mucus, blood, saliva, urine, sputum, or cell scraping sample.

In some embodiments, the animal is human. In certain embodiments, the test sample comprises an analyte. In some embodiments, the analyte is a protein or nucleic acid. In certain embodiments, the nucleic acid is RNA or DNA. In some embodiments, the analyte is RNA. In some embodiments, the analyte is DNA. In some embodiments, the analyte is a protein. In certain embodiments, the analyte is a protein. In some embodiments, the analyte is an antibody or fragment thereof.

In some embodiments, the composition is for use in a fluidic device, diagnostic test system, personal care product, cosmetic product, cleaning product, foaming agent, pharmaceutical application, oil-recovery application, or agricultural protection application.

In some embodiments, the composition is for use in a fluidic device. In some embodiments, the composition is for use in a diagnostic test system. In some embodiments, the composition is for use in a personal care product. In some embodiments, the composition is for use in a personal cosmetic product. In some embodiments, the composition is for use in a cleaning product. In some embodiments, the composition is for use in a foaming agent. In some embodiments, the composition is for use in a pharmaceutical application. In some embodiments, the composition is for use in an oil-recovery application. In some embodiments, the composition is for use in an agricultural protection application.

In one aspect, the present disclosure provides a method of detecting a target nucleic acid sequence, the method comprising:
a) performing a nucleic acid amplification reaction configured to amplify the target nucleic acid sequence using a composition that includes the compound disclosed herein; and
b) detecting the presence or absence of the target nucleic acid sequence.

In some embodiments, the target nucleic acid sequence is obtained from a biological sample from a subject. In certain embodiments, the target nucleic acid sequence is a DNA sequence or an RNA sequence. In certain embodiments, the target nucleic acid sequence is a DNA sequence. In certain embodiments, the target nucleic acid sequence is an RNA sequence. In certain embodiments, the target nucleic acid sequence is a DNA sequence, and wherein the nucleic acid amplification reaction comprises LAMP.

In some embodiments, the subject is a human, non-human primate, or mouse subject.

In some embodiments, the target nucleic acid sequence is an RNA sequence, and wherein the nucleic acid amplification reaction comprises RT-LAMP.

In certain embodiments, the target nucleic acid sequence is detected using a lateral flow assay (LFA) strip, a colorimetric assay, a fluorescence assay, an electrochemical method of detection, a CRISPR/Cas method of detection, or is directly detected using hybridization. In certain embodiments, the target nucleic acid sequence is detected using a colorimetric assay. In certain embodiments, the target nucleic acid sequence is detected using a fluorescence assay. In certain embodiments, the target nucleic acid sequence is detected using an electrochemical method of detection. In certain embodiments, the target nucleic acid sequence is detected using a CRISPR/Cas method of detection. In certain embodiments, the target nucleic acid sequence is directly detected using hybridization.

In some embodiments, the biological sample comprises mucus, saliva, sputum, urine, blood, or cell scraping sample. In some embodiments, the biological sample comprises mucus. In some embodiments, the biological sample comprises saliva. In some embodiments, the biological sample comprises sputum. In some embodiments, the biological sample comprises urine. In some embodiments, the biological sample comprises blood. In some embodiments, the biological sample comprises a cell scraping sample.

In some embodiments, the compounds of present disclosure can be used to improve fluid dynamics of a device. In some embodiments, the improvement of fluid dynamics comprises increased control of flow direction and rate. In some embodiments, the improved fluid dynamics is further characterized by increased control of flow direction or rate.

In certain embodiments, provided is a method of reducing the surface tension of a solution, the method comprising adding to the solution the compound described herein.

In some embodiments, provided is a method of decreasing viscosity of a solution, the method comprising adding to the solution the compound disclosed herein.

In some embodiments, the method further comprises increased wettability.

In certain embodiments, provided is a method of inducing emulsification of an analyte in a solution, the method comprising adding to the solution the compound described herein.

In some embodiments, provided is a method of increasing solubility of an analyte in a solution, the method comprising adding to the solution the compound described herein or the composition of the present disclosure.

In certain embodiments, the analyte comprises dirt.

In some embodiments, the analyte comprises oil.

In certain embodiments, the analyte comprises an active pharmaceutical agent.

In some embodiments, provided is a method of improving nucleic acid detection in an assay, the method comprising adding to the assay the compound of the present disclosure or the composition described herein.

In one aspect, the compounds of the present disclosure may be used in a method of analyzing a biological sample.

In another aspect, provided is kit for the detection of a target nucleic acid sequence comprising a compound disclosed herein.

Also encompassed by the disclosure are kits. The kits provided may comprise a compound or composition described herein and a container (e.g., a vial, ampule, bottle, syringe, and/or dispenser package, or other suitable container). In some embodiments, provided kits may optionally further include a second container comprising an excipient for dilution or suspension of the composition or compound described herein.

Thus, in one aspect, provided are kits including a first container comprising a compound or composition described herein. In certain embodiments, the kits are useful for improving fluid dynamics of a system (i.e., decreasing surface tension, increasing wettability, inducing emulsification).

In certain embodiments, a kit described herein further includes instructions for using the kit. A kit described herein may also include information as required by a regulatory agency such as the U.S. Food and Drug Administration (FDA).

### EMBODIMENTS

Embodiment 1. A compound of Formula (I):

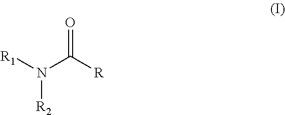

wherein:
   R is substituted or unsubstituted aliphatic;
   R¹ is H or optionally-substituted polyethylene glycol; and
   R² is H or optionally-substituted polyethylene glycol;
   provided that at least one of R¹ and R² is not H.

Embodiment 2. The compound of Embodiment 1, wherein R is substituted or unsubstituted alkyl.

Embodiment 3. The compound of Embodiment 1, wherein R is branched or unbranched alkyl.

Embodiment 4. The compound of any one of Embodiments 1-2, wherein:
R is

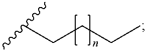

wherein n is 1-20.

Embodiment 5. The compound of any one of Embodiments 1-2, wherein:
R is

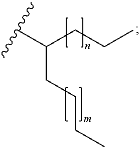

wherein
n is 1-20; and
m is 1-20.

Embodiment 6. The compound of any one of Embodiments 1-2, wherein:
R is

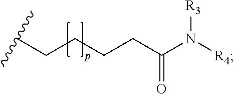

wherein
R₃ is H or optionally-substituted polyethylene glycol; and
R₄ is H or optionally-substituted polyethylene glycol;
provided that at least one of R₃ and R₄ is not H; and
p is 1-20.

Embodiment 7. The compound of any one of Embodiments 1-6, wherein the optionally-substituted polyethylene glycol is of the formula:

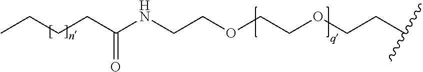

wherein
   n′ is 1-20; and
   q′ is 1-20.

Embodiment 8. The compound of any one of Embodiments 1-7, selected from formulae:

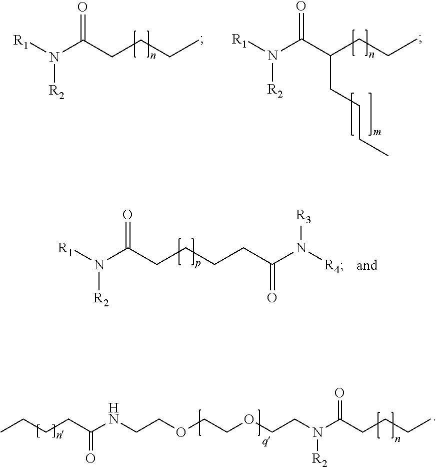

Embodiment 9. The compound of Embodiment 4, of the formula:

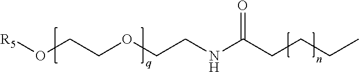

wherein
   R₅ is methyl (CH₃)
   q is 1-20.

Embodiment 10. The compound of Embodiment 4, of the formula:

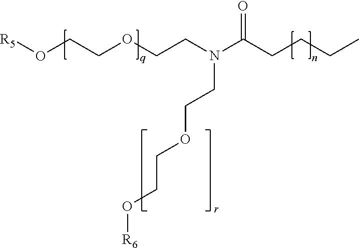

wherein
   R₅ is methyl (CH₃);
   R₆ is methyl (CH₃);
   q is 1-20;
   r is 1-20.

Embodiment 11. The compound of Embodiment 5, of the formula:

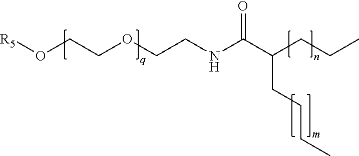

wherein
   R₅ is methyl (CH₃); and
   q is 1-20.

Embodiment 12. The compound of Embodiment 5, of the formula:

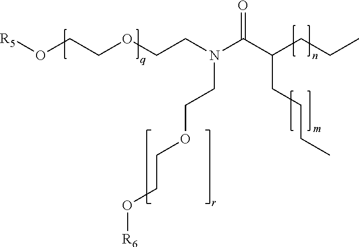

wherein
   R₅ is methyl (CH₃);
   R₆ is methyl (CH₃);
   q is 1-20;
   r is 1-20.

Embodiment 13. The compound of Embodiment 6, of the formula:

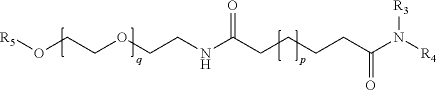

wherein
   R₅ is methyl (CH₃); and
   q is 1-20.

Embodiment 14. The compound of Embodiment 13, of the formula:

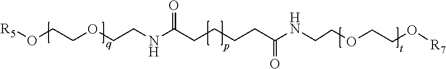

wherein
   R₇ is methyl (CH₃); and
t is 1-20.

Embodiment 15. The compound of Embodiment 13, of the formula

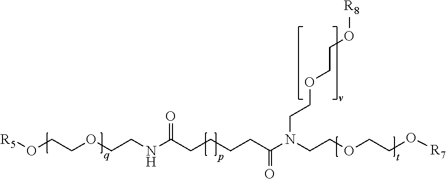

wherein
R₇ is methyl (CH₃);
R₈ is methyl (CH₃);
t is 1-20; and
v is 1-20.

Embodiment 16. The compound of Embodiment 6, of the formula:

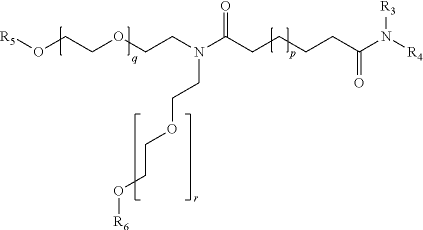

wherein
   R₅ is methyl (CH₃);
   R₆ is methyl (CH₃);
   q is 1-20;
   r is 1-20.

Embodiment 17. The compound of Embodiment 16, of the formula:

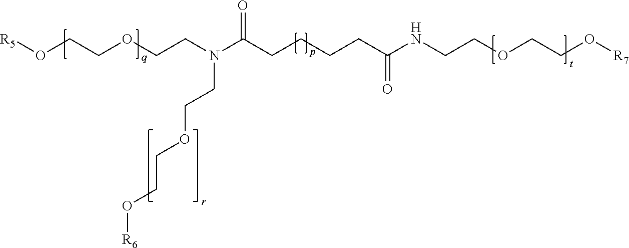

wherein
   R₇ is methyl (CH₃); and
   t is 1-20.

Embodiment 18. The compound of Embodiment 16, of the formula:

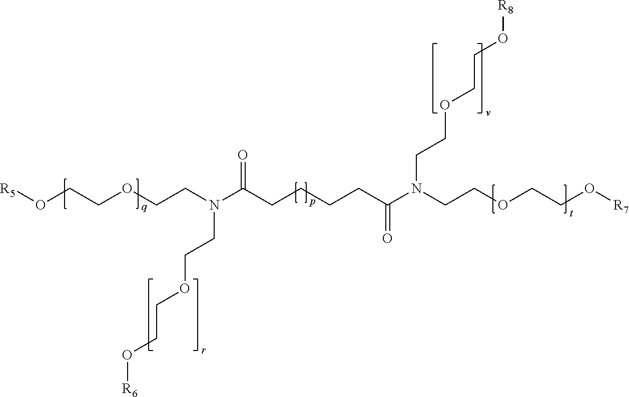

wherein
   R₇ is methyl (CH₃);
   R₈ is methyl (CH₃);
   t is 1-20; and
   v is 1-20.

Embodiment 19. The compound of Embodiment 1, selected from:

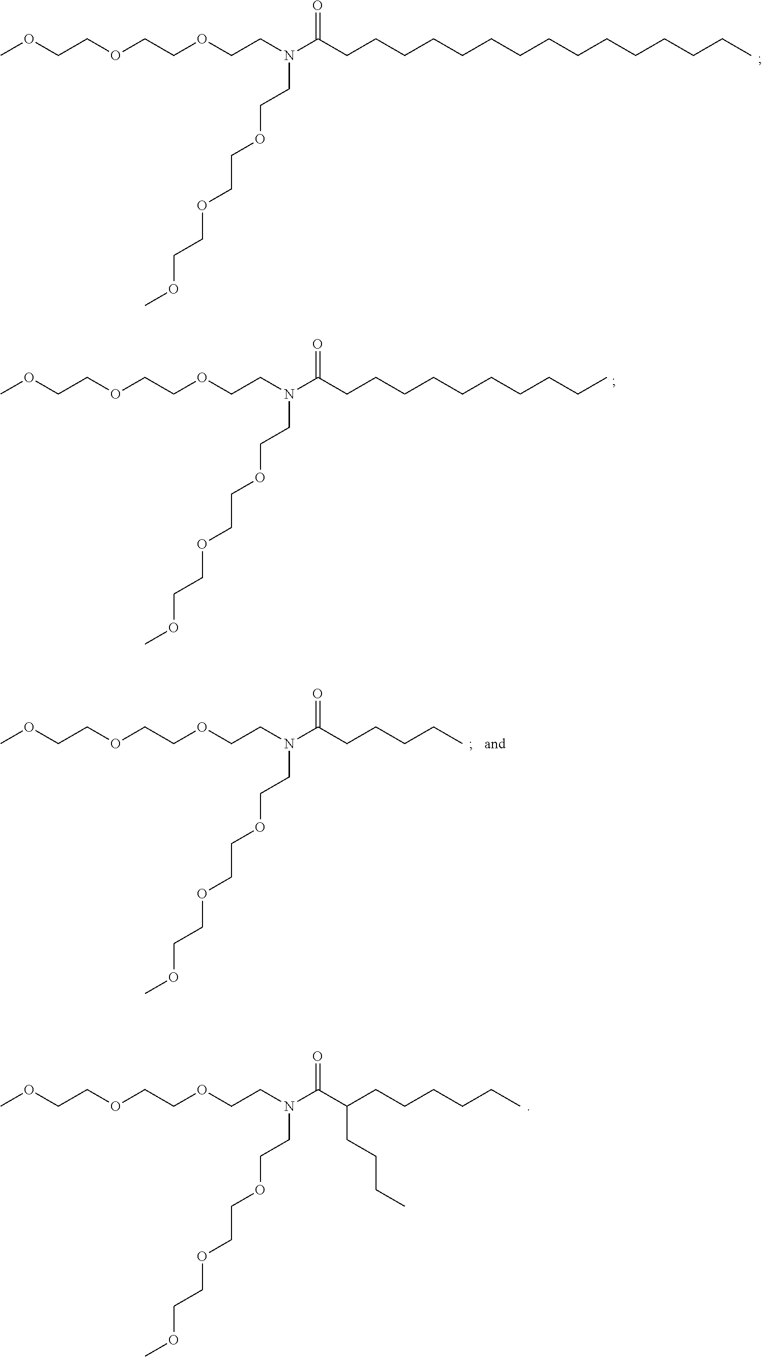

Embodiment 20. The compound of any one of Embodiments 1-19, wherein the compound is stable at room temperature, for example, exhibiting a main peak purity of ≥80% following 1-12 months of storage at room temperature.

Embodiment 21. The compound of any one of Embodiments 1-20, wherein the compound is compatible with nucleic acid amplification reactions, including but not limited to loop mediated isothermal amplification and associated biological reagents, such as polymerase and reverse transcriptase.

Embodiment 22. A composition comprising the compound of any one of Embodiments 1-21.

Embodiment 23. The composition of Embodiment 22, characterized as being monodisperse.

Embodiment 24. The composition of any one of Embodiments 22-23, further comprising a biological molecule, such as a protein or oligonucleotide.

Embodiment 25. The composition of any one of Embodiments 22-23, for use in a fluidic device.

Embodiment 26. The composition of any one of Embodiments 22-23, for use in a diagnostic test system.

Embodiment 27. The composition of any one of Embodiments 22-23, for use in a personal care product or cosmetic application.

Embodiment 28. The composition of any one of Embodiments 22-23, for use in a cleaning product.

Embodiment 29. The composition of any one of Embodiments 22-23, for use in a pharmaceutical application.

Embodiment 30. The composition of any one of Embodiments 22-23, for use in oil-recovery application.

Embodiment 31. The composition of any one of Embodiments 22-23, for use in agricultural protection applications.

Embodiment 32. A method of analyzing a biological sample, comprising preparing a solution comprising the biological sample and a compound of any one of Embodiments 1-21, and analyzing said solution.

Embodiment 33. A method of detecting a target nucleic acid sequence, the method comprising:
(i) obtaining a biological sample from a subject;
(ii) performing a nucleic acid amplification reaction configured to amplify the target nucleic acid sequence using a composition that includes the compound of any one of Embodiments 1-21 and
(iii) detecting the presence of absence of the target nucleic acid sequence

Embodiment 34. The method of Embodiment 33, wherein the target nucleic acid sequence is a DNA sequence or an RNA sequence.

Embodiment 35. The method of Embodiment 33 or Embodiment 34, wherein the subject is a human, non-human primate, or mouse subject.

Embodiment 36. The method of any one of Embodiments 33-35, wherein the target nucleic acid sequence is a DNA sequence, and wherein the nucleic acid amplification reaction comprises LAMP.

Embodiment 37. The method of any one of Embodiments 33-35, wherein the target nucleic acid sequence is an RNA sequence, and wherein the nucleic acid amplification reaction comprises RT-LAMP.

Embodiment 38. The method of any one of Embodiments 33-37, wherein the target nucleic acid sequence is detected using a lateral flow assay (LFA) strip, a colorimetric assay, a fluorescence assay, an electrochemical method of detection, a CRISPR/Cas method of detection, or is directly detected using hybridization.

Embodiment 39. The method of any one of Embodiments 33-38, wherein the biological sample comprises a mucus, saliva, sputum, urine, blood, or cell scraping sample.

Embodiment 40. A kit for the detection of a target nucleic acid sequence comprising a compound of any one of Embodiments 1-21.

Embodiment 41. A method of making a kit for the detection of a target nucleic acid sequence comprising a compound according to any one of Embodiments 1-21.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1. Synthesis of DS1

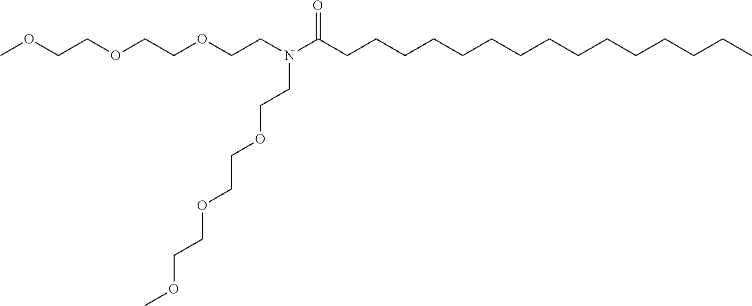 [SMILES: CCCCCCCCCCCCCCCC(=O)N(CCOCCOCCOC)CCOCCOCCOC]

To a solution of bis(2-(2-(2-methoxyethoxy)ethoxy)ethyl)amine (200 mg, 0.646 mmol) in MeCN (2.00 mL) was added triethylamine (0.128 mL, 0.918 mmol) at room temperature and stirred for 5 min, followed by addition of palmitoyl chloride (0.168 mL, 0.614 mmol). The resulting reaction mixture was stirred for 40 min. The reaction mixture was quenched with H₂O and directly purified by reversed phase chromatography (0 to 100% MeCN/H₂O 0.1% TFA, RediSep Rf Gold® 50 g HP C₁₈ column). Evaporation of the product containing fractions afforded the desired product (272 mg, 81% yield) as a colorless oil. HRMS (ESI) calculated for C₃₀H₆₂NO₇⁺ (M+H)⁺ 548.4521, observed 548.4532.

### Example 2. Synthesis of DS2

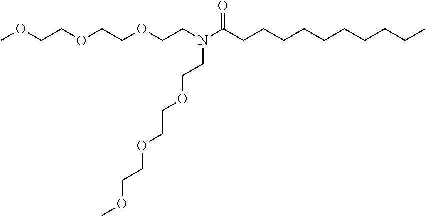 [SMILES: CCCCCCCCCCC(=O)N(CCOCCOCCOC)CCOCCOCCOC]

To a solution of bis(2-(2-(2-methoxyethoxy)ethoxy)ethyl)amine (196 mg, 0.633 mmol) in MeCN (3.00 mL) was added triethylamine (0.13 mL, 0.932 mmol) at room temperature and stirred for 5 min, followed by addition of undecanoyl chloride (0.15 mL, 0.681 mmol). The resulting reaction mixture was stirred for 30 min. The reaction mixture was quenched with H₂O and directly purified by reversed phase chromatography (0 to 100% MeCN/H₂O, RediSep Rf Gold® 50 g HP C₁₈ column). Evaporation of the product containing fractions afforded the desired product (258 mg, 85% yield) as a light yellow oil. HRMS (ESI) calculated for C₂₅H₅₂NO₇⁺ (M+H)⁺ 478.3738, observed 478.3756.

### Example 3. Synthesis of DS3

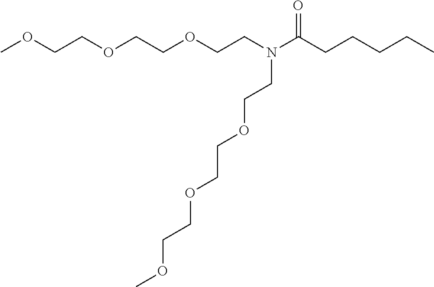 [SMILES: CCCCCC(=O)N(CCOCCOCCOC)CCOCCOCCOC]

To a solution of bis(2-(2-(2-methoxyethoxy)ethoxy)ethyl)amine (246 mg, 0.795 mmol) in MeCN (2.00 mL) was added triethylamine (0.128 mL, 0.918 mmol) at room temperature and stirred for 5 min, followed by addition of hexanoyl chloride (0.1 mL, 0.715 mmol). The resulting reaction mixture was stirred for 30 min. The reaction mixture was quenched with H₂O and directly purified by reversed phase chromatography (0 to 100% MeCN/H₂O, RediSep Rf Gold® 50 g HP C₁₈ column). Evaporation of the product containing fractions afforded the desired product (280 mg, 96% yield) as a colorless oil. HRMS (ESI) calculated for C₂₀H₄₂NO₇⁺ (M+H)⁺ 408.2956, observed 408.2968.\

### Example 4. Synthesis of DS4

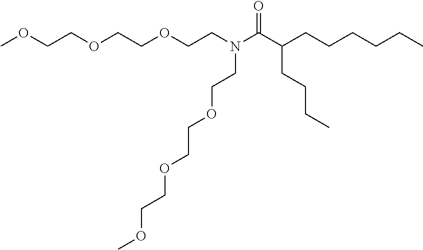 [SMILES: CCCCCCC(CCCC)C(=O)N(CCOCCOCCOC)CCOCCOCCOC]

To a solution of bis(2-(2-(2-methoxyethoxy)ethoxy)ethyl)amine (192 mg, 0.621 mmol) in MeCN (3.00 mL) was added triethylamine (0.130 mL, 0.933 mmol) at room temperature and stirred for 5 min, followed by addition of 2-butyloctanoyl chloride (0.143 g, 0.654 mmol). The resulting reaction mixture was stirred for 1 h. The reaction mixture was quenched with H₂O and directly purified by reversed phase chromatography (0 to 100% MeOH/H₂O, RediSep Rf Gold® 50 g HP C₁₈ column). Evaporation of the product containing fractions afforded the desired product (268 mg, 88% yield) as a light-yellow oil. HRMS (ESI) calculated for C₂₆H₅₃NNaO₇⁺ (M+Na)⁺ 514.3714, observed 514.3723.

### Example 5. Synthesis of DS5

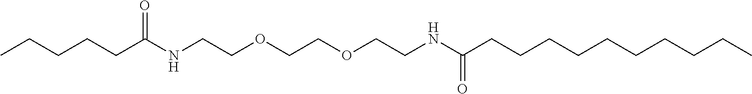 [SMILES: CCCCCCCCCCC(=O)NCCOCCOCCNC(=O)CCCCC]

To a solution of tert-butyl (2-(2-(2-aminoethoxy)ethoxy)ethyl) carbamate (0.3 mL, 1.26 mmol) in MeCN (4.00 mL) was added triethylamine (0.26 mL, 1.87 mmol) at room temperature and stirred for 5 min, followed by addition of undecanoyl chloride (0.28 mL, 1.27 mmol). The resulting reaction mixture was stirred for 1 h. The reaction mixture was quenched with H₂O and directly purified by reversed phase chromatography (0 to 100% MeOH/H₂O, RediSep Rf Gold® 50 g HP C₁₈ column). The intermediate compound was taken straight to the next reaction step.

To a chilled solution of tert-butyl (2-(2-(2-undecanamidoethoxy)ethoxy)ethyl) carbamate (532 mg, 1.28 mmol) in CH₂Cl₂ (3 mL) at 0° C. was added TFA (1 mL, 13.1 mmol) dropwise over 1 min. The resulting reaction mixture was stirred for 2 h, followed by evaporation and trituration with Et₂O. The crude mixture was then dissolved in MeCN (5 mL) and added triethylamine (0.54 mL, 3.87 mmol) at room temperature and stirred for 5 min, followed by addition of undecanoyl chloride (0.19 mL, 1.36 mmol). The resulting reaction mixture was stirred for 1 h. The reaction mixture was quenched with H₂O and directly purified by reversed phase chromatography (0 to 100% MeOH/H₂O, RediSep Rf Gold® 50 g HP C₁₈ column). Evaporation of the product containing fractions afforded the desired product (368 mg, 69% yield) as a white solid (DS5). HRMS (ESI) calculated for C₂₃H₄₇N₂O₄⁺ (M+H)⁺ 415.3530, observed 415.3536.

### Example 6. Synthesis of DS6

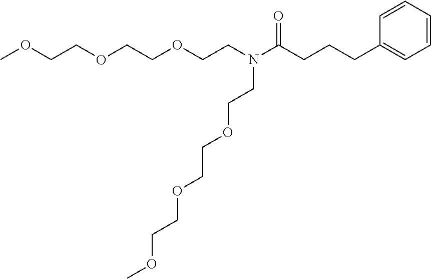 [SMILES: COCCOCCOCCN(CCOCCOCCOC)C(=O)CCCc1ccccc1]

To a solution of bis(2-(2-(2-methoxyethoxy)ethoxy)ethyl)amine (197 mg, 0.637 mmol) in MeCN (3 mL) was added triethylamine (0.13 mL, 0.932 mmol) at room temperature and stirred for 5 min, followed by addition of 4-phenylbutanoyl chloride (0.11 mL, 0.671 mmol). The resulting reaction mixture was stirred for 30 min. The reaction mixture was quenched with H₂O and directly purified by reversed phase chromatography (0 to 100% MeOH/H₂O, RediSep Rf Gold® 50 g HP C₁₈ column). Evaporation of the product containing fractions afforded the desired product (174 mg, 60% yield) as a light-yellow oil (DS6). HRMS (ESI) calculated for C₂₄H₄₂NO₇⁺ (M+H)⁺ 456.2956, observed 456.2973.

### Example 7. Synthesis of DS7

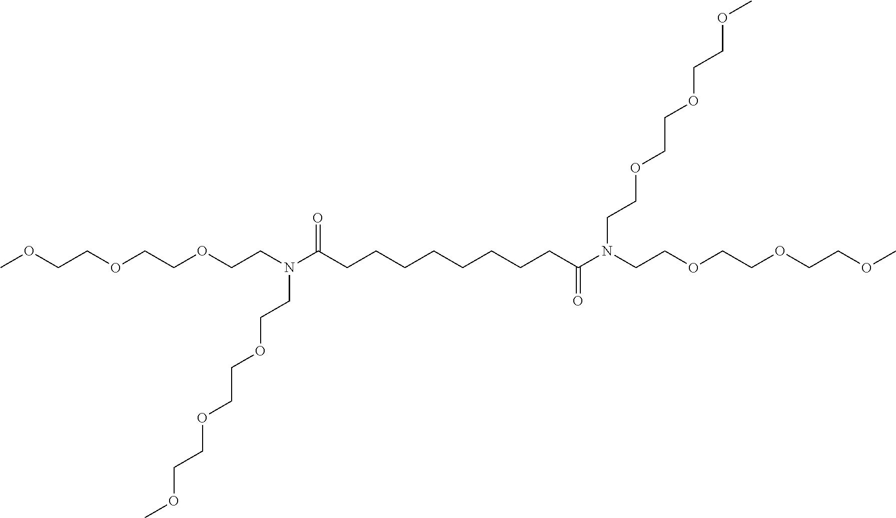

To a solution of bis(2,5-dioxopyrrolidin-1-yl) decanedioate (239 mg, 0.603 mmol) in MeCN (4 mL) was added bis(2-(2-(2-methoxyethoxy)ethoxy)ethyl)amine (410 mg, 1.33 mmol), followed by addition of triethylamine (0.18 mL, 1.29 mmol) at room temperature. The resulting reaction mixture was stirred for 1 h. The reaction mixture was quenched with H₂O and directly purified by reversed phase chromatography (0 to 100% MeOH/H₂O, RediSep Rf Gold® 50 g HP C₁₈ column). Evaporation of the product containing fractions afforded the desired product (212 mg, 45% yield) as a light brown oil (DS7). HRMS (ESI) calculated for C₃₈H₇₇N₂O₁₄⁺ (M+H)⁺ 785.5369, observed 785.5396.

### Example 8. Synthesis of DS9

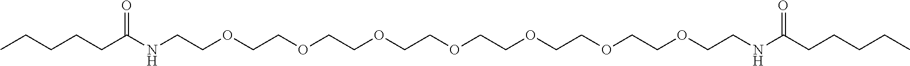 [SMILES: CCCCCC(=O)NCCOCCOCCOCCOCCOCCOCCOCCNC(=O)CCCCC]

To a solution of 3,6,9,12,15,18,21-heptaoxatricosane-1,23-diamine (216 mg, 0.586 mmol) in MeCN (4 mL) was added triethylamine (0.18 mL, 1.29 mmol) at room temperature and stirred for 5 min, followed by addition of hexanoyl chloride (0.2 mL, 1.43 mmol). The resulting reaction mixture was stirred for 2 h. The reaction mixture was quenched with H₂O and directly purified by reversed phase chromatography (0 to 100% MeCN/H₂O, RediSep Rf Gold® 50 g HP C₁₈ column). Evaporation of the product containing fractions afforded the desired product (237 mg, 72% yield) as a white solid (DS9). HRMS (ESI) calculated for C₂₈H₅₇N₂O₉⁺ (M+H)⁺ 565.4059, observed 565.4080.

### Example 9. Synthesis of DS10

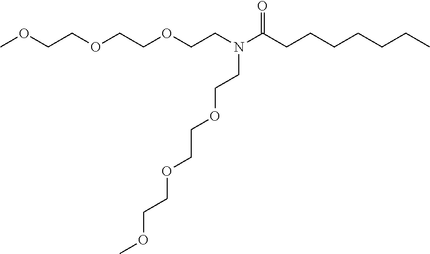 [SMILES: CCCCCCCC(=O)N(CCOCCOCCOC)CCOCCOCCOC]

To a solution of bis(2-(2-(2-methoxyethoxy)ethoxy)ethyl)amine (190 mg, 0.614 mmol) in MeCN (3 mL) was added triethylamine (0.12 mL, 0.861 mmol) at room temperature and stirred for 5 min, followed by addition of 2,5-dioxopyrrolidin-1-yl octanoate (141 mg, 0.584 mmol). The resulting reaction mixture was stirred for 2 h. The reaction mixture was quenched with H₂O and directly purified by reversed phase chromatography (0 to 100% MeOH/H₂O, RediSep Rf Gold® 50 g HP C₁₈ column). Evaporation of the product containing fractions afforded the desired product (112 mg, 44% yield) as a light-yellow oil (DS10). HRMS (ESI) calculated for C₂₂H₄₆NO₇⁺ (M+H)⁺ 436.3269, observed 436.3286.

### Example 10. Synthesis of DS11

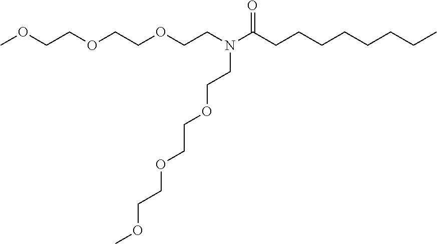 [SMILES: CCCCCCCCC(=O)N(CCOCCOCCOC)CCOCCOCCOC]

To a solution of bis(2-(2-(2-methoxyethoxy)ethoxy)ethyl)amine (150 mg, 0.485 mmol) in MeCN (2 mL) was added triethylamine (0.10 mL, 0.717 mmol) at room temperature and stirred for 5 min, followed by addition of 2,5-dioxopyrrolidin-1-yl nonanoate (118 mg, 0.462 mmol). The resulting reaction mixture was stirred for 2 h. The reaction mixture was quenched with H₂O and directly purified by reversed phase chromatography (0 to 100% MeOH/H₂O, RediSep Rf Gold® 50 g HP C₁₈ column). Evaporation of the product containing fractions afforded the desired product (126 mg, 61% yield) as a light-yellow oil (DS11). HRMS (ESI) calculated for C₂₃H₄₈NO₇⁺ (M+H)⁺ 450.3425, observed 450.3441.

### Example 11. Synthesis of DS12

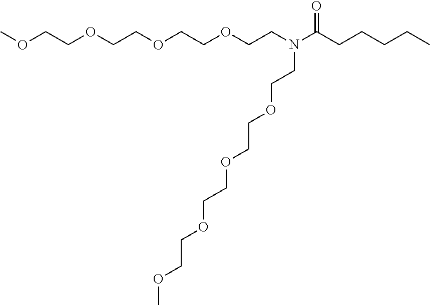 [SMILES: CCCCCC(=O)N(CCOCCOCCOCCOC)CCOCCOCCOCCOC]

To a solution of di(2,5,8,11-tetraoxatridecan-13-yl)amine (212 mg, 0.533 mmol) in MeCN (3 mL) was added triethylamine (0.10 mL, 0.717 mmol) at room temperature and stirred for 5 min, followed by addition of hexanoyl chloride (0.10 mL, 0.715 mmol). The resulting reaction mixture was stirred for 40 min. The reaction mixture was quenched with H₂O and directly purified by reversed phase chromatography (0 to 100% MeCN/H₂O, RediSep Rf Gold® 50 g HP C₁₈ column). Evaporation of the product containing fractions afforded the desired product (135 mg, 51% yield) as a colorless oil (DS12). HRMS (ESI) calculated for C₂₄H₅₀NO₉⁺ (M+H)⁺ 496.3480, observed 496.3496.

### Example 12. Synthesis of DS 113

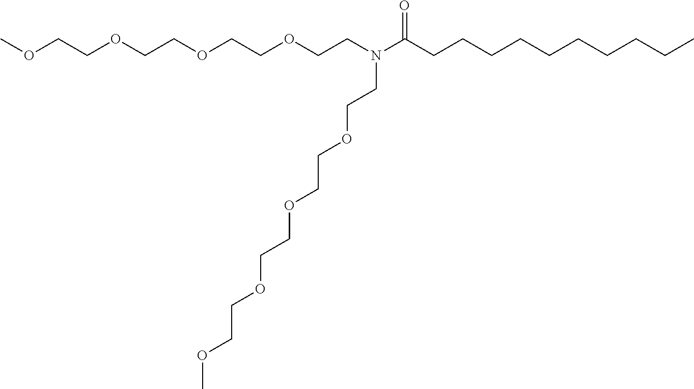 [SMILES: CCCCCCCCCCC(=O)N(CCOCCOCCOCCOC)CCOCCOCCOCCOC]

To a solution of di(2,5,8,11-tetraoxatridecan-13-yl)amine (193 mg, 0.486 mmol) in MeCN (3 mL) was added triethylamine (0.10 mL, 0.717 mmol) at room temperature and stirred for 5 min, followed by addition of undecanoyl chloride (0.12 mL, 0.545 mmol). The resulting reaction mixture was stirred for 40 min. The reaction mixture was quenched with H₂O and directly purified by reversed phase chromatography (0 to 100% MeCN/H₂O, RediSep Rf Gold® 50 g HP C₁₈ column). Evaporation of the product containing fractions afforded the desired product (185 mg, 67% yield) as a light-yellow oil (DS13). HRMS (ESI) calculated for C₂₉H₆₀NO₉⁺ (M+H)⁺ 566.4263, observed 566.4281.

### Example 13. Synthesis of DS14

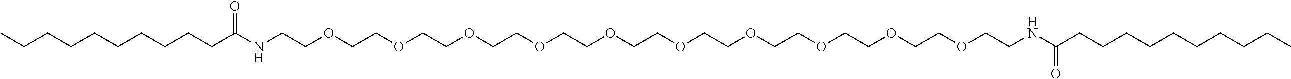 [SMILES: CCCCCCCCCCC(=O)NCCOCCOCCOCCOCCOCCOCCOCCOCCOCCOCCNC(=O)CCCCCCCCCC]

To a solution of 3,6,9,12,15,18,21,24,27,30-decaoxadotriacontane-1,32-diamine (82 mg, 0.164 mmol) in MeCN (2 mL) was added triethylamine (60 μL, 0.430 mmol) at room temperature and stirred for 5 min, followed by addition of undecanoyl chloride (80 μL, 0.363 mmol). The resulting reaction mixture was stirred for 2 h. The reaction mixture was quenched with H₂O and directly purified by reversed phase chromatography (0 to 100% MeOH/H₂O, RediSep Rf Gold® 50 g HP C₁₈ column). Evaporation of the product containing fractions afforded the desired product (109 mg, 80% yield) as a white solid (DS14). HRMS (ESI) calculated for C₄₄H₈₉N₂O₁₂⁺ (M+H)⁺ 837.6410, observed 837.6438.

### Example 14. Synthesis of DS15

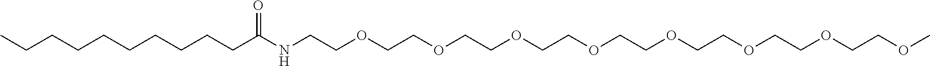 [SMILES: CCCCCCCCCCC(=O)NCCOCCOCCOCCOCCOCCOCCOCCOC]

To a solution of 2,5,8,11,14,17,20,23-octaoxapentacosan-25-amine (207 mg, 0.539 mmol) in MeCN (3 mL) was added triethylamine (0.11 mL, 0.79 mmol) at room temperature and stirred for 5 min, followed by addition of undecanoyl chloride (0.12 mL, 0.545 mmol). The resulting reaction mixture was stirred for 30 min. The reaction mixture was quenched with H₂O and directly purified by reversed phase chromatography (0 to 100% MeOH/H₂O, RediSep Rf Gold® 50 g HP C₁₈ column). Evaporation of the product containing fractions afforded the desired product (204 mg, 69% yield) as a light-yellow oil (DS15). HRMS (ESI) calculated for C₂₈H₅₇NNaO₉⁺ (M+Na)⁺ 574.3926, observed 574.3942.

### Example 15. Synthesis of DS16

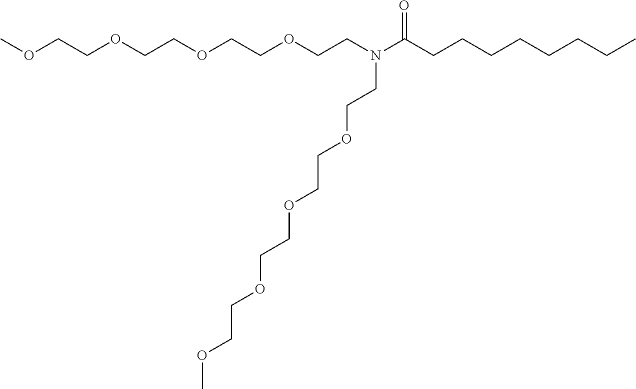 [SMILES: CCCCCCCCC(=O)N(CCOCCOCCOCCOC)CCOCCOCCOCCOC]

To a solution of di(2,5,8,11-tetraoxatridecan-13-yl)amine (180 mg, 0.453 mmol) in MeCN (2 mL) was added triethylamine (90 μL, 0.646 mmol) at room temperature and stirred for 5 min, followed by addition of 2,5-dioxopyrrolidin-1-yl nonanoate (110 mg, 0.431 mmol). The resulting reaction mixture was stirred for 2 h. The reaction mixture was quenched with H₂O and directly purified by reversed phase chromatography (0 to 100% MeOH/H₂O, RediSep Rf Gold® 50 g HP C₁₈ column). Evaporation of the product containing fractions afforded the desired product (99.4 mg, 43% yield) as a light-yellow oil (DS16). HRMS (ESI) calculated for C₂₇H₅₆NO₉⁺ (M+H)⁺ 538.3950, observed 538.3971.

### Example 16. Synthesis of DS17

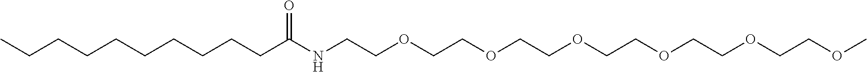 [SMILES: CCCCCCCCCCC(=O)NCCOCCOCCOCCOCCOCCOC]

To a solution of 2,5,8,11,14,17-hexaoxanonadecan-19-amine (500 mg, 1.69 mmol) in MeCN (5 mL) was added triethylamine (0.35 mL, 2.51 mmol) at room temperature and stirred for 5 min, followed by addition of undecanoyl chloride (0.39 mL, 1.77 mmol). The resulting reaction mixture was stirred for 30 min. The reaction mixture was quenched with H₂O and directly purified by reversed phase chromatography (0 to 100% MeOH/H₂O, RediSep Rf Gold® 50 g HP C₁₈ column). Evaporation of the product containing fractions afforded the desired product (346 mg, 44% yield) as a light-yellow oil (DS17). HRMS (ESI) calculated for C₂₄H₄₉NNaO₇⁺ (M+Na)⁺ 486.3401, observed 486.3420.

### Example 17. Synthesis of DS18

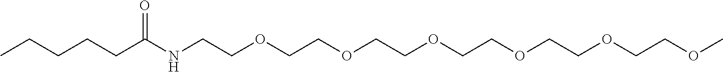 [SMILES: CCCCCC(=O)NCCOCCOCCOCCOCCOCCOC]

To a solution of 2,5,8,11,14,17-hexaoxanonadecan-19-amine (500 mg, 1.69 mmol) in MeCN (5 mL) was added triethylamine (0.35 mL, 2.51 mmol) at room temperature and stirred for 5 min, followed by addition of hexanoyl chloride (0.25 mL, 1.79 mmol). The resulting reaction mixture was stirred for 30 min. The reaction mixture was quenched with H₂O and directly purified by reversed phase chromatography (0 to 100% MeOH/H₂O, RediSep Rf Gold® 50 g HP C₁₈ column). Evaporation of the product containing fractions afforded the desired product (364 mg, 55% yield) as a light-yellow oil (DS18). HRMS (ESI) calculated for C₁₉H₃₉NNaO₇⁺ (M+Na)⁺ 416.2619, observed 416.2632.

### Example 18. DetectSurfactants (DS) are Compatible with LAMP Reactions

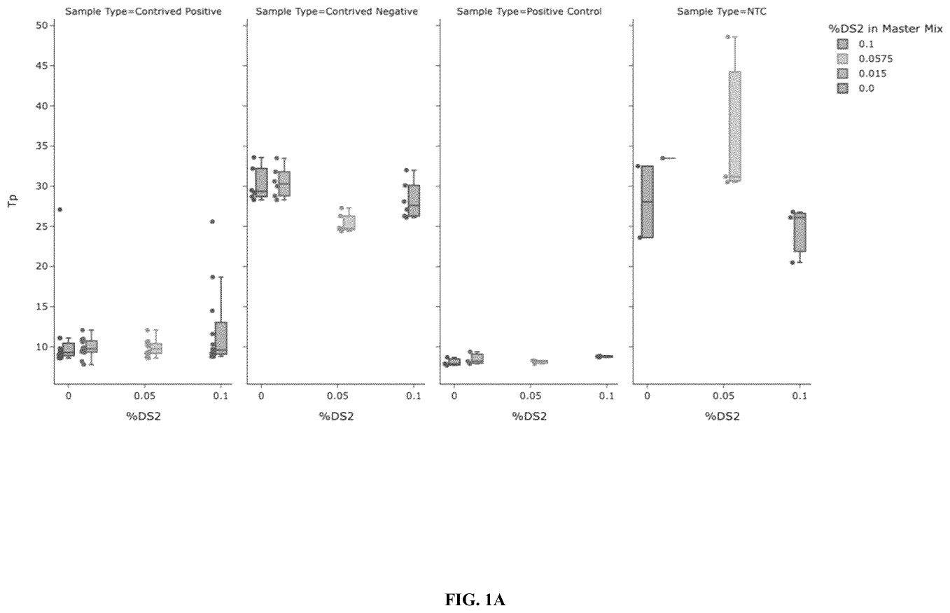

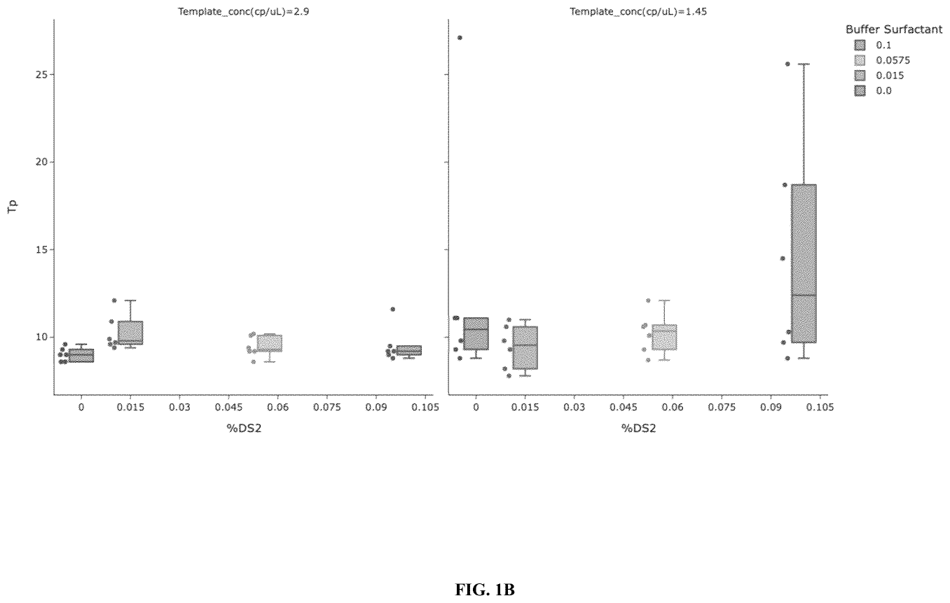

Titration of DS2 in a FluB LAMP reaction and in the presence of nasal matrix (FIGS. 1A-B). DS2 was added at 0%, 0.015%, 0.0575%, and 0.1% to the LAMP MasterMix, or LAMP buffer, containing target-specific primers, dNTPs, DNA polymerase, reverse transcriptase, intercalating fluorescent dye, and the target RNA sequence. This reaction mixture was incubated at 62° C. for 50 minutes and the increase in fluorescence was assessed to determine the time to positivity.

As the surfactant concentration approaches its critical micelle concentration (CMC), detrimental impact is observed on LAMP performance. For example, at the CMC value of 0.1% of DS2, there is a delay in the contrived positive amplification, as well as impact on the non-template control (NTC)(FIG. 1A). This results in worse overall specificity. However, at concentrations below the CMC, there is no detrimental impact on assay specificity.

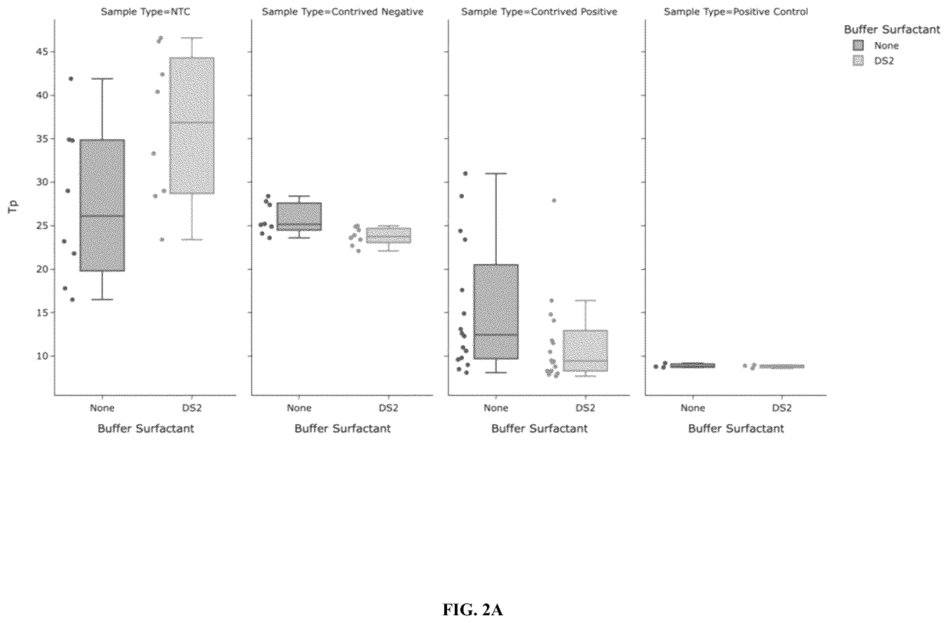

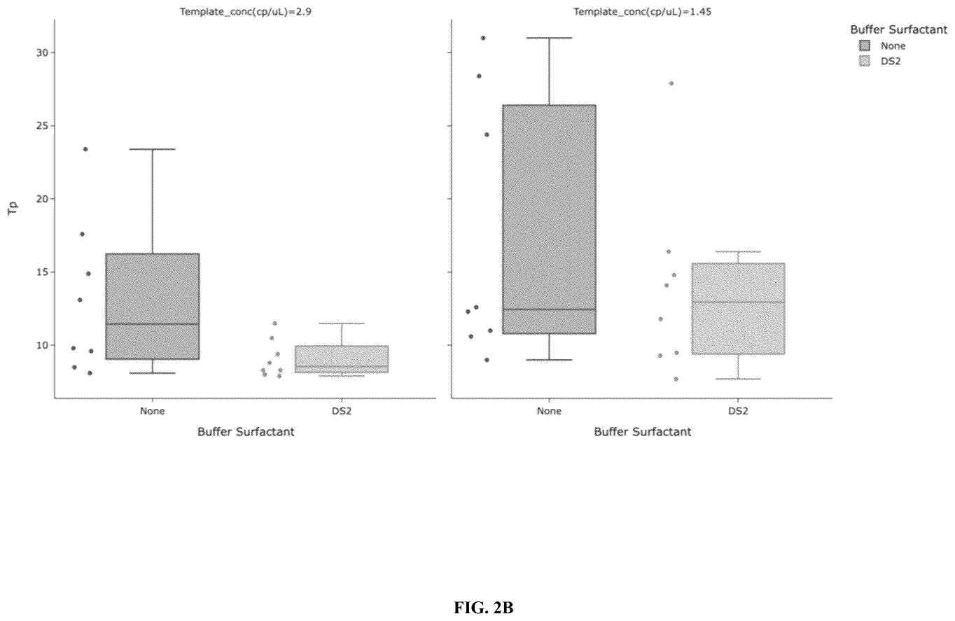

DS2 was added at 0.015% to the LAMP buffer and in the presence of nasal matrix (FIGS. 2A-B). It was determined that DS2 is compatible with FluB assay in the presence of nasal matrix at 0.015%.

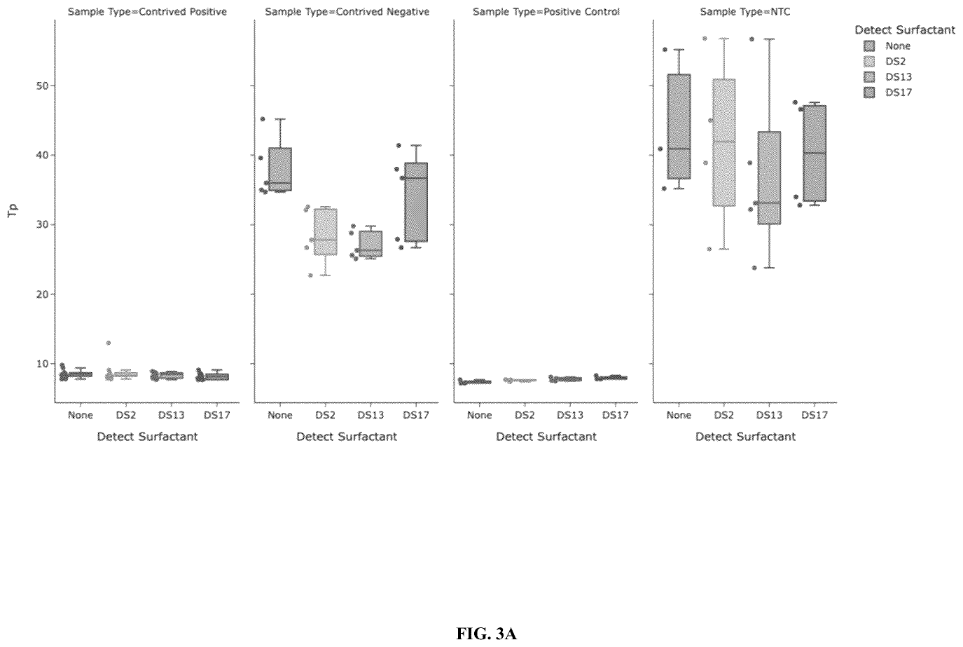

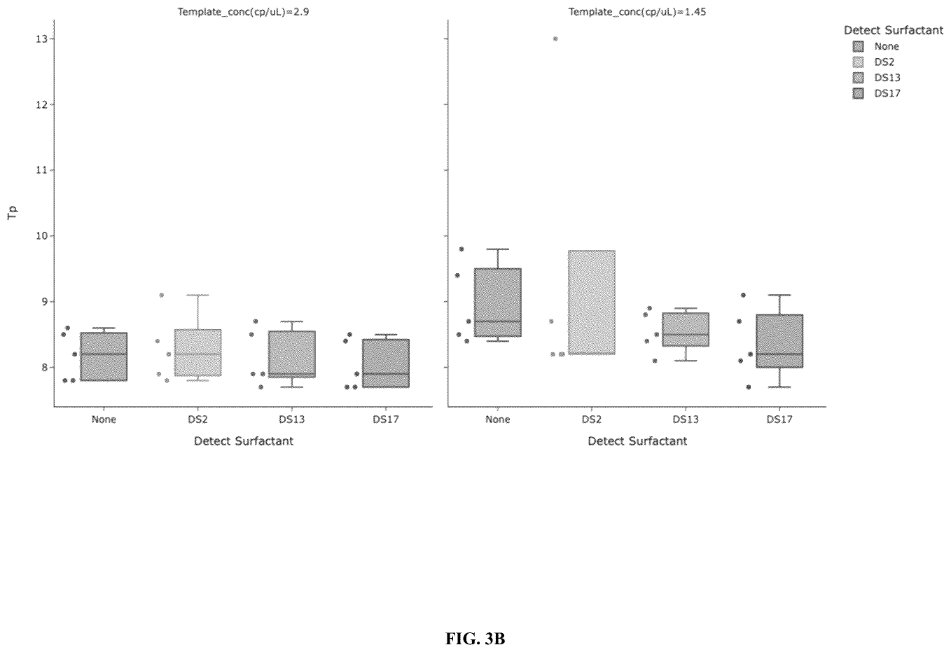

DS2, DS13, and DS17 were added at 0.0075% to the LAMP MasterMix in the presence of nasal matrix (FIGS. 3A-B). It was determined that DS2, DS13, and DS17 is compatible with FluB assay in the presence of nasal matrix at 0.0075%.

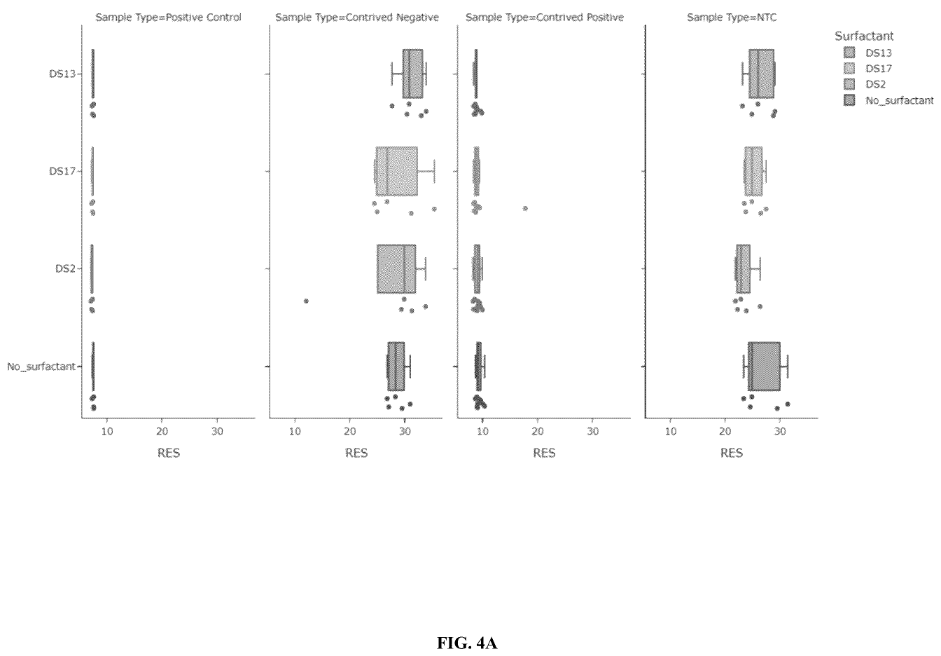

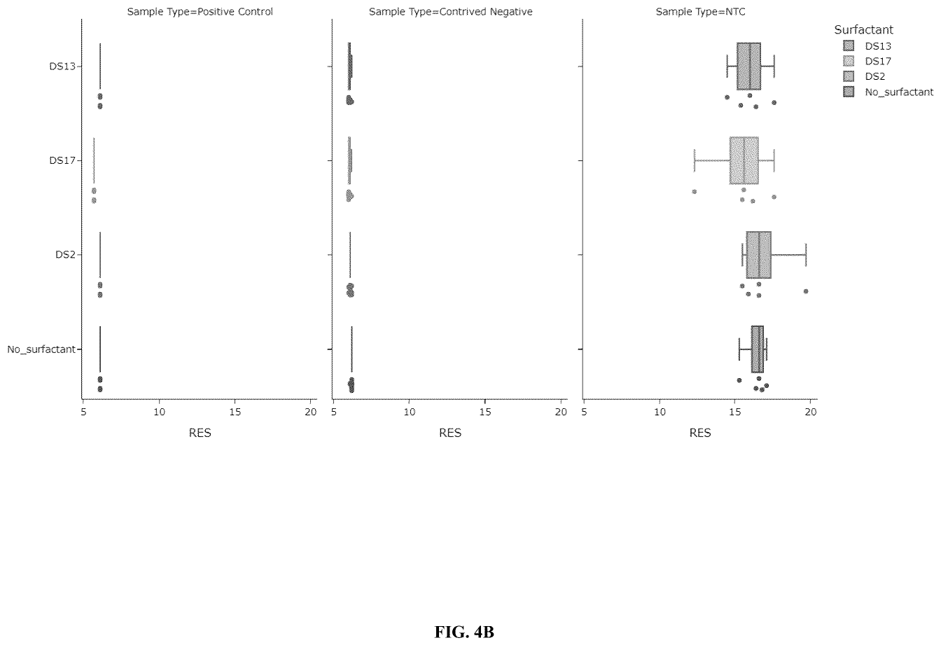

DS2, DS13, and DS17 were added at 0.0075% to the LAMP MasterMix using the above procedure with SARS-COV-2 target template (FIG. 4A). It was determined that DS2, DS13, and DS17 is compatible with FluB assay in in a SARS-COV-2 assay at 0.0075%.

DS2, DS13, and DS17 were added at 0.0075% to the LAMP MasterMix using the above procedure with Ex2B target template (FIG. 4B). It was determined that DS2, DS13, and DS17 is compatible with FluB assay in in a Ex2B assay at 0.0075%.

### Example 19. DSs are Stable Stored in Buffer

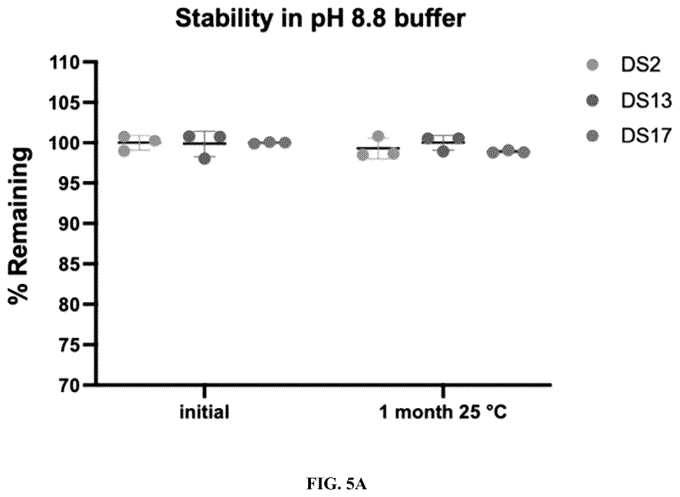

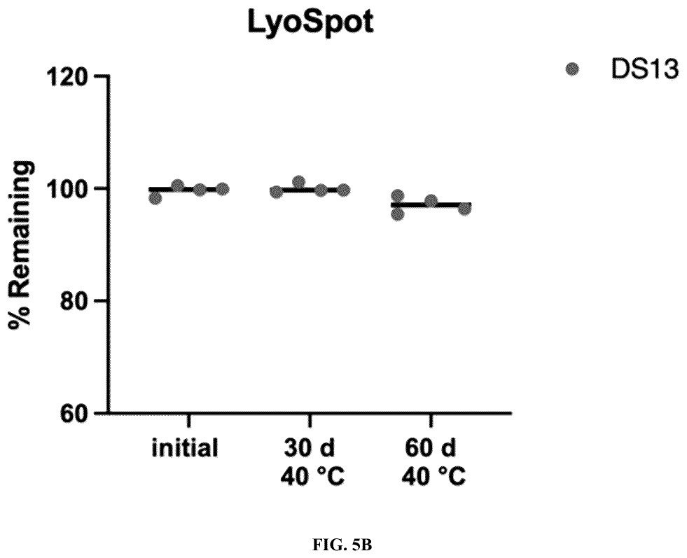

Solutions of isothermal amplification buffers at pH 8.8 containing 0.1% of DS2, DS13, or DS17 were kept at 25° C. and analyzed over time using HPLC. It was observed that 95%-100% of the surfactant remained after one month (FIG. 5A) and therefore DSs are stable in pH 8.8 amplification buffer when stored in ambient conditions.

DS 13 was subject to accelerated stability conditions (40° C.) with lyophilized material for one and two months (FIG. 5B). At each time point, material was dissolved in water and analyzed by HPLC. As assayed by HPLC, DS13 was found to be stable under these conditions at the 2-month time point (97.1±1.5% remaining), corresponding to 4 months stability at room temperature (15-30° C.).

### Example 20. DSs Improve Physical Appearance of Lyophilized Material of LAMP Components

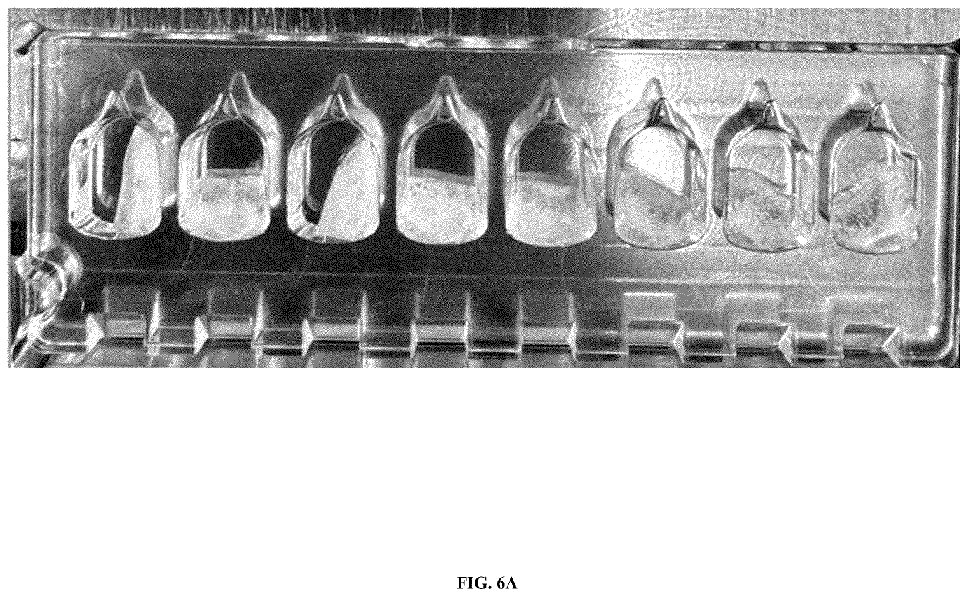

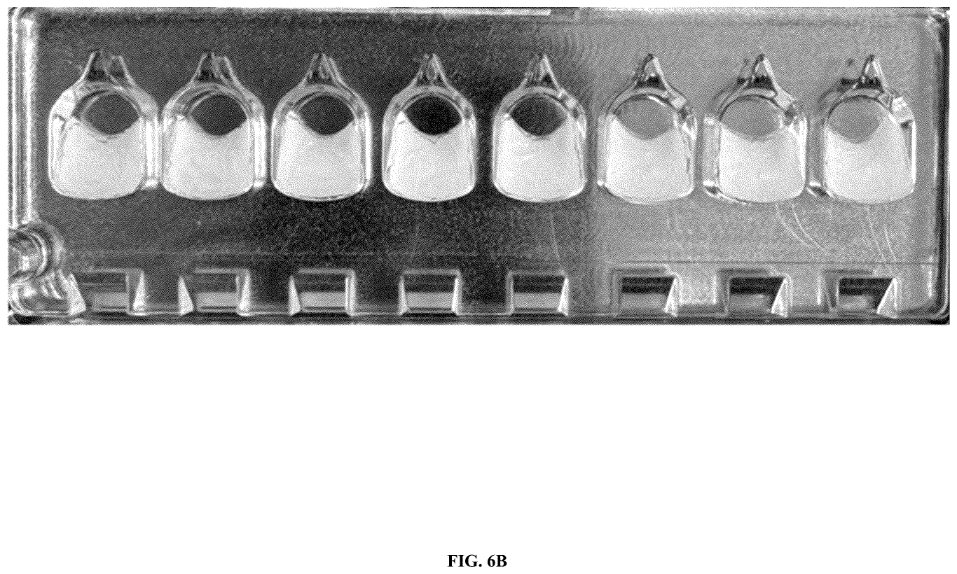

Lyospots (containing dNTP, dye, enzymes, primers, excipients, and buffer) with (FIG. 6B) and without (FIG. 6A) 0.06% DS13 were assessed based on visual appearance. Lyospots formulated with DS 13 result in a more consistent shape and amorphous physical appearance on plastic substrates by reduction in the surface tension.

### Example 21. Lyophilization of MS2 LAMP Chemistry

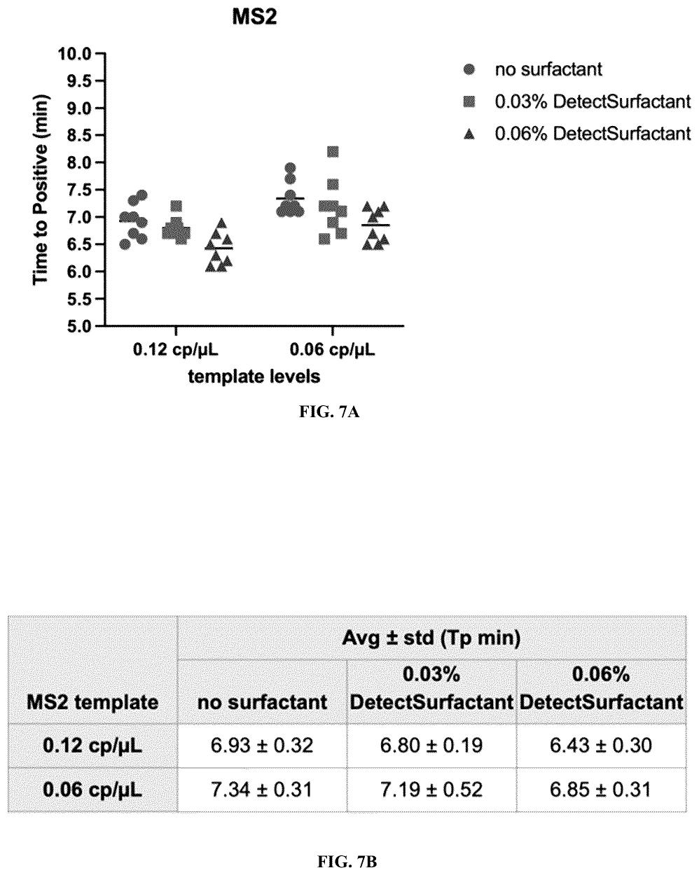

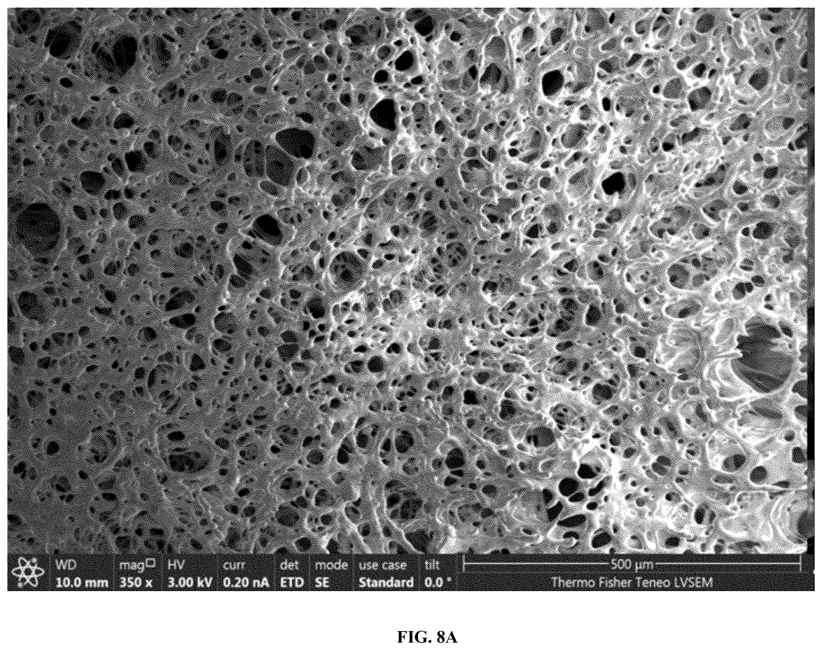

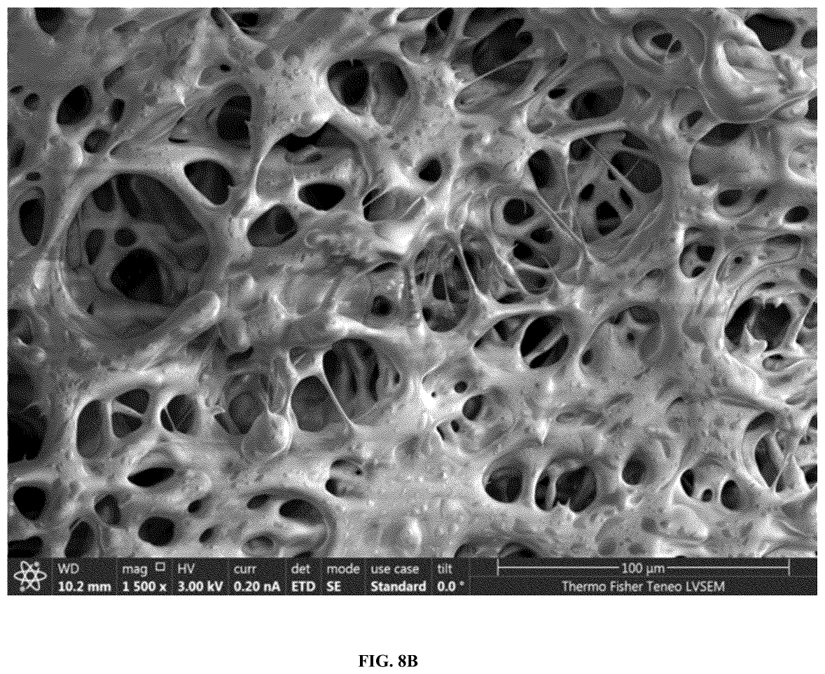

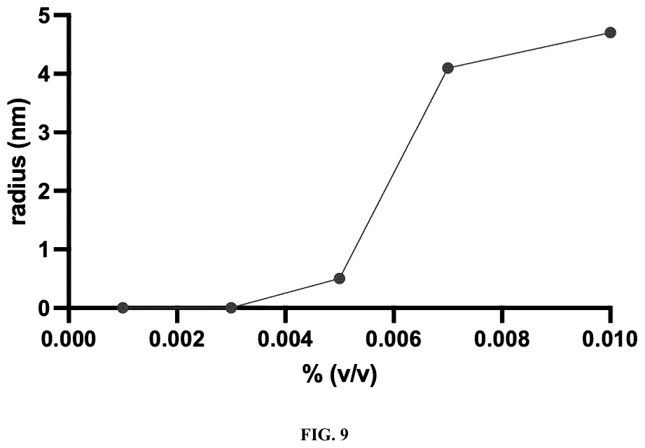

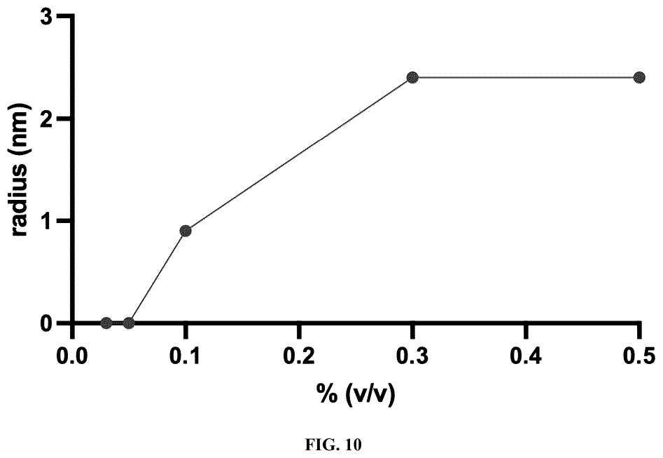

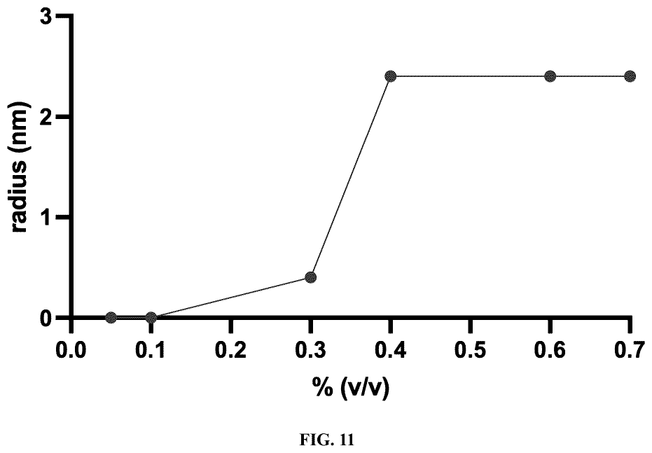

Lyophilization of MS2 LAMP chemistry at different levels of DS 13 (0%, 0.03%, or 0.06%), resulted in no detrimental impact on the time to positive (Tp, min) at low template inputs (0.12 cp/μL and 0.06 cp/μL)(FIGS. 7A-B). SEM images of lyophilized material with DS13 indicate that the lyophilized material consists of mainly open pores (FIGS. 8A-B).

### Example 22. CMC Determination

Average micelle radius data was collected on a DynaPro NanoStar Dynamic Light Scattering Detector. To each disposable cuvette was added 5 μL of the specified solution of DS1, DS2, or DS13 in water (T=25° C., n=10).

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included.

Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 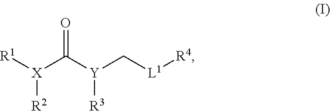
or a salt thereof, wherein:
X and Y are each independently C or N,
R¹ optionally substituted alkyl, or optionally substituted polyethylene glycol;
L¹ is optionally substituted alkylene, optionally wherein one or more backbone carbon atoms in the optionally substituted alkylene are independently replaced with a moiety selected from —O—, —NR₅C(═O)—, —C(═O)NR₅—, or —NR₅—;
R² and R³ are each independently hydrogen, optionally substituted alkyl, or optionally substituted polyethylene glycol;
R⁴ is hydrogen, optionally substituted alkyl, polyethylene glycol, or optionally substituted aryl; and
R₅ is, independently for each instance, hydrogen, optionally substituted alkyl, optionally substituted acyl, optionally substituted polyethylene glycol, or a nitrogen protecting group;
wherein the compound of formula (I) comprises at least one optionally substituted polyethylene glycol.

2. (canceled)

3. The compound of claim 1, of formula (I-a): 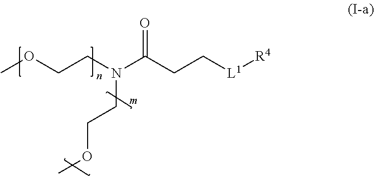
or a salt thereof, wherein:
n and m are each independently 1-20.

4-7. (canceled)

8. The compound of claim 3, or a salt thereof, wherein the compound is

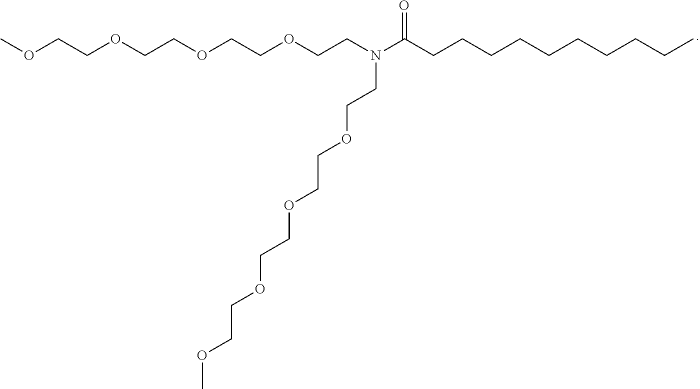 [SMILES: CCCCCCCCCCC(=O)N(CCOCCOCCOCCOC)CCOCCOCCOCCOC]

9. The compound of claim 3, of formula (I-a-V): 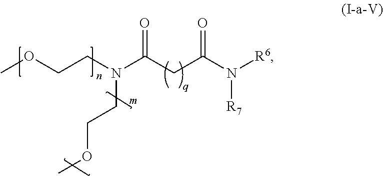
or a salt thereof, wherein:
R⁶ and R⁷ are each optionally substituted polyethylene glycol; and
q is 1-20.

10. The compound of claim 1, of formula (I-b): 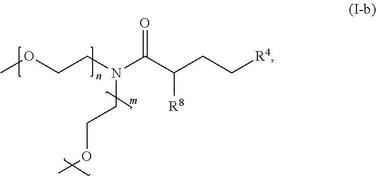
or a salt thereof, wherein:
R⁸ is optionally substituted C₁₋₂₀ alkyl.

11. (canceled)

12. The compound of claim 1, of formula (I-c): 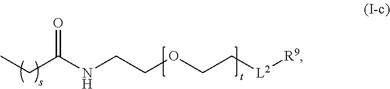
or a salt thereof, wherein:
s and t are each independently 1-20;
L² is a bond or optionally substituted alkylene, optionally wherein one or more backbone carbon atoms in the optionally substituted alkylene are independently replaced with a moiety selected from —O—, —NR₅C(═O)—, —C(═O)NR₅—, or —NR₅—; and
R⁹ is hydrogen, methyl, or optionally substituted aryl.

13. The compound of claim 12, of formula (I-c-I): 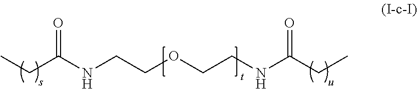
or a salt thereof, wherein:
u is 1-20;
t is 1-10; and
s is 1-10.

14-16. (canceled)

17. The compound of claim 12, of formula (I-c-II): 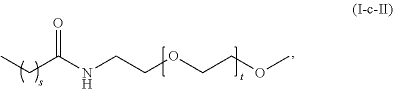
or a salt thereof, wherein
s is 1-10; and
t is 1-10.

18-21. (canceled)

22. The compound of claim 12, of formula (I-c-III): 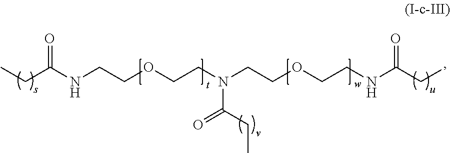
or a salt thereof, wherein:
u, v, and w are each independently 1-20.

23-24. (canceled)

25. The compound of claim 1, wherein the compound is:

| Compound | Structure |
|---|---|
| DS1 | 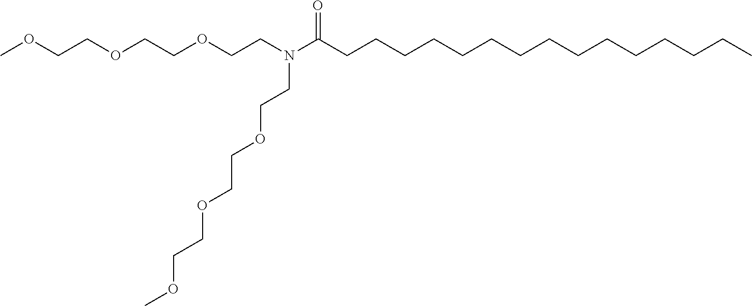 [SMILES: CCCCCCCCCCCCCCCC(=O)N(CCOCCOCCOC)CCOCCOCCOC] |
| DS2 | 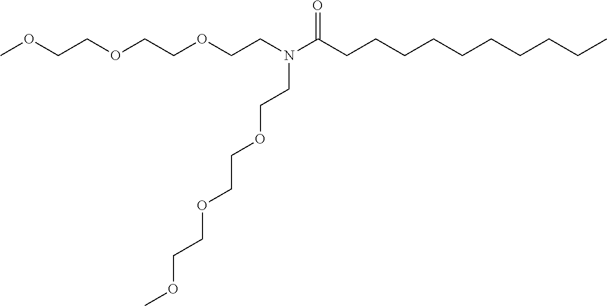 [SMILES: CCCCCCCCCCC(=O)N(CCOCCOCCOC)CCOCCOCCOC] |
| DS3 | 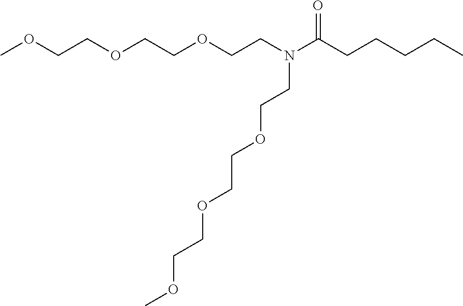 [SMILES: CCCCCC(=O)N(CCOCCOCCOC)CCOCCOCCOC] |
| DS4 | 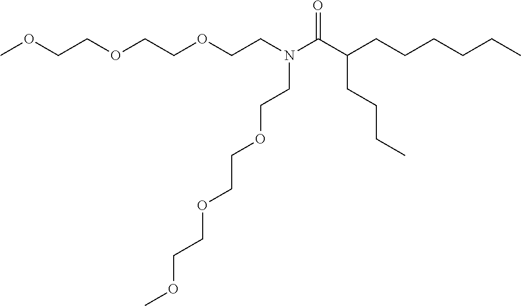 [SMILES: CCCCCCC(CCCC)C(=O)N(CCOCCOCCOC)CCOCCOCCOC] |
| DS5 | 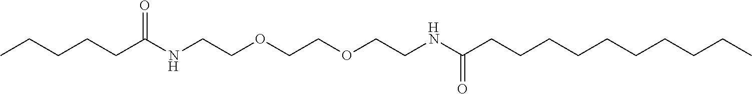 |
| DS6 | 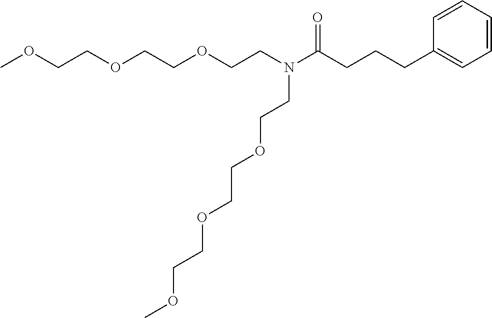 [SMILES: COCCOCCOCCN(CCOCCOCCOC)C(=O)CCCc1ccccc1] |
| DS7 | 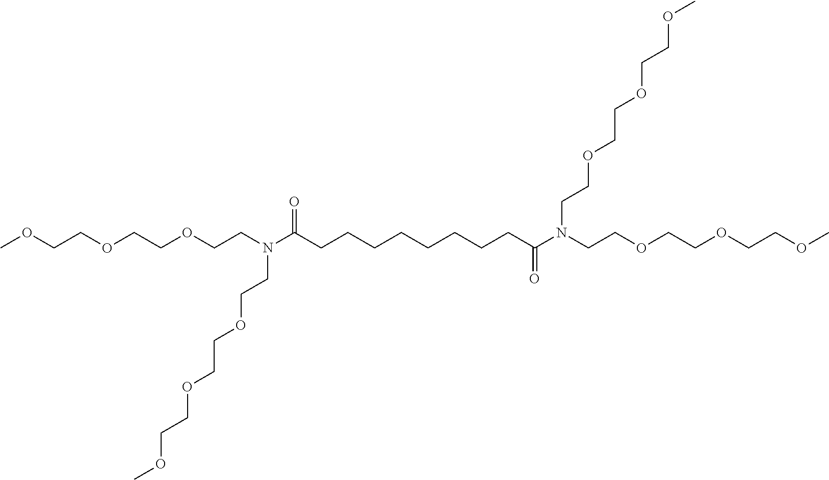 |
| DS8 | |
| DS9 | 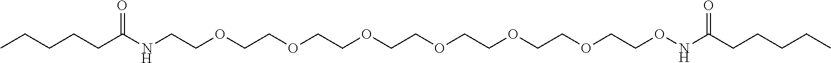 |
| DS10 | 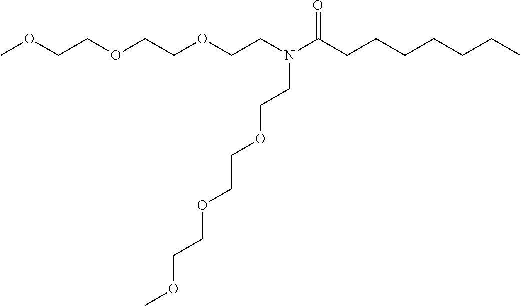 [SMILES: CCCCCCCC(=O)N(CCOCCOCCOC)CCOCCOCCOC] |
| DS11 | 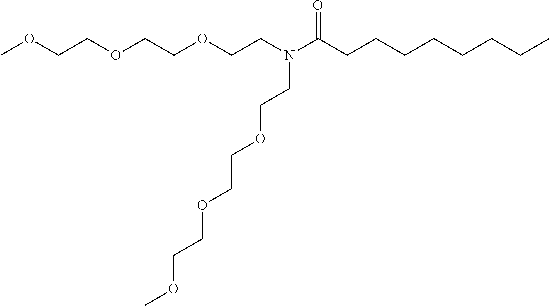 [SMILES: CCCCCCCCC(=O)N(CCOCCOCCOC)CCOCCOCCOC] |
| DS12 | 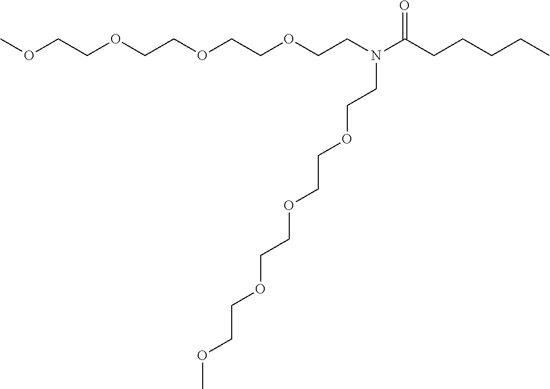 [SMILES: CCCCCC(=O)N(CCOCCOCCOCCOC)CCOCCOCCOCCOC] |
| DS13 | 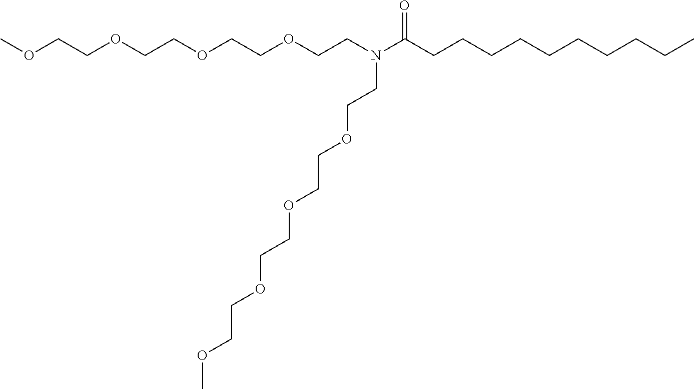 [SMILES: CCCCCCCCCCC(=O)N(CCOCCOCCOCCOC)CCOCCOCCOCCOC] |
| DS14 | |
| DS15 | |
| DS16 | 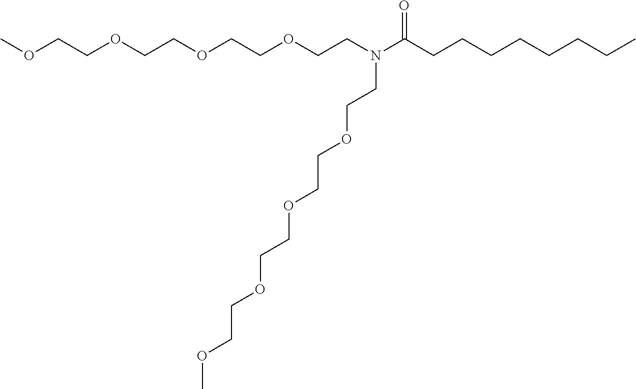 [SMILES: CCCCCCCCC(=O)N(CCOCCOCCOCCOC)CCOCCOCCOCCOC] |
| DS17 | |
| DS18. | |

26. (canceled)

27. The compound of claim 1, or a salt thereof, wherein the compound has a critical micelle concentration (CMC) of 0.005-0.5%.

28-29. (canceled)

30. A composition comprising the compound of claim 1.

31. (canceled)

32. The composition of claim 30, further comprising a test sample, wherein the test sample comprises an analyte.

33-35. (canceled)

36. The composition of claim 32, wherein the analyte is a protein or nucleic acid.

37. (canceled)

38. The composition of claim 30, for use in a fluidic device, diagnostic test system, personal care product, cosmetic product, cleaning product, foaming agent, pharmaceutical application, oil-recovery application, or agricultural protection application.

39. A method of detecting a target nucleic acid sequence, the method comprising:
a) performing a nucleic acid amplification reaction configured to amplify the target nucleic acid sequence using a composition that comprises the compound of claim 1; and
b) detecting the presence or absence of the target nucleic acid sequence.

40. The method of claim 39, wherein the target nucleic acid sequence is obtained from a biological sample comprising mucus, saliva, sputum, urine, blood, or cell scraping from a subject.

41-42. (canceled)

43. The method of claim 39, wherein the target nucleic acid sequence is a DNA sequence or an RNA sequence, and wherein the nucleic acid amplification reaction comprises LAMP.

44. (canceled)

45. The method of claim 39, wherein the target nucleic acid sequence is detected using a lateral flow assay (LFA) strip, a colorimetric assay, a fluorescence assay, an electrochemical method of detection, a CRISPR/Cas method of detection, or is directly detected using hybridization.

46. (canceled)

47. A kit for the detection of a target nucleic acid sequence comprising the compound of claim 1.
